(19) United States
(12) Patent Application Publication
(10) Pub. No.: US 2002/0169102 A1
(43) Pub. Date: Nov. 14, 2002

(54) Intranasal delivery of agents for regulating development of implanted cells in the CNS

(76) Inventor: William H. Frey II, White Bear, MN (US)

Correspondence Address: Chiron Corporation, Intellectual Property, P.O. Box 8097, Emeryville, CA, 94608-2916, US

(21) Appl. No.: 10/114,385

(22) Filed: Apr. 2, 2002

Related U.S. Application Data
(60) Provisional application No. 60/281,062, filed on Apr. 3, 2001.

(51) Int. Cl. A61K 31/00; C12N 5/08

(52) U.S. Cl. 514/1; 435/368

(57) ABSTRACT

The present invention provides a method of regulating the development of a donor cell in the central nervous system of a mammal. The method comprises administering a composition comprising a therapeutically effective amount of at least one regulatory agent, preferably a growth factor such as bFGF, NGF, or IGF-I, or an agent that modulates the immune response to a tissue of the mammal innervated by the trigeminal nerve and/or the olfactory nerve. The methods find use in improving the clinical outcome of a mammal having undergone a neural regenerative strategy. Hence, the present invention is directed to the treatment and/or prevention of CNS disorders, such as, epilepsy, stroke, ischemia, Huntington disease, Parkinson's disease, ALS, Alzheimer's disease, brain and spinal cord injuries and demyelinating or dysmyelinating disorders, such as Pelizaeus-Merzbacher disease and multiple sclerosis.

### CROSS-REFERENCE TO RELATED APPLICATIONS

This application claims the benefit of U.S. Provisional Application No. 60/281,062, filed Apr. 3, 2001, which is hereby incorporated herein in its entirety by reference.

### FIELD OF THE INVENTION

The present invention is directed to a method for delivering a regulatory agent by way of a tissue innervated by the trigeminal nerve and/or olfactory nerve to the central nervous system of a mammal having undergone a neural regenerative strategy.

### BACKGROUND OF THE INVENTION

Many neurological conditions result from the loss of certain cell populations from the nervous system through disease or injury. The cells destroyed in these conditions are not intrinsically replaced. Recent evidence demonstrates that neuronal replacement and partial reconstruction of neuronal circuitry is possible via cell transplantation therapies. Much of the initial work in the field used fetal-cell therapies. In recent years, however, it has become evident that the developing and even the adult mammalian nervous system contains a population of undifferentiated, multipotent, neural stem cells that display plastic properties that are advantageous for the design of more effective neural regenerative strategies for many of these neurological conditions.

For example, in Parkinson's disease, the neurons that degenerate comprise the dopaminergic neurons of the substantia nigra. Current cell replacement strategies for patients with advanced Parkinson's disease comprise intrastriatal grafts of nigral dopaminergic neurons from 6- to 9-week-old human embryos. Clinical improvements develop gradually over the first 6-24 months after transplantation (Olanow et al. (1996) Trends Neurosci. 19:102-109 and Lindvall et al. (1999) Mov. Disord. 14:201-205). Further, the first study of human fetus-to-adult striatal transplantation has recently been performed in three nondemented patients with moderately advanced Huntington disease. Magnetic resonance imaging evaluation at 1 year documented graft survival and growth without displacement of surrounding tissue. All patients improved on some measure of cognitive function, although no uniform pattern was evident (Kopyov et al. (1998) J. Exp. Neurol. 149:97-108). See also, Date et al. (1997) J. Exp. Neurol. 147:10-17.

Neural stem cells have also been demonstrated to replace lost and dying cells and lost neural circuits in the degenerating CNS. For instance, treatment of mice with MPTP, a drug that selectively destroys dopaminergic cells in the brain stem, followed by grafting with a neural stem cell population, resulted in a reconstituted dopaminergic cell population composed of both donor and host cells. Similar studies in mice using a hypoxia-ischemic brain injury model showed transplantation of neural stem cells enhanced the recovery of the damaged system (Park et al. (1999) J. Neurotrauma 16:675-687 and Park et al. (1997) Soc. Neurosci. Abst. 23:346).

Regulatory factors such as various growth factors including insulin-like growth factor-I (IGF-I), nerve growth factor (NGF), and basic fibroblast growth factor (bFGF), regulate the survival and differentiation of nerve cells during the development of the peripheral and central nervous systems. Neurotrophins are also required for nerve growth during development (Tucker et al. (2001) Nature Neurosci. 4:29-37). In the mature nervous system, these trophic factors maintain the morphologic and neurochemical characteristics of nerve cells and strengthen functionally active synaptic connections. Such regulatory factors find use in enhancing the methods of neural cell replacement strategies.

For instance, bFGF enhances survival and growth of neurons in vitro. Further, bFGF produces a potent growth promoting effect on implanted neurons in vivo when the implanted neurons are genetically engineered to express the bFGF (Takayama et al. (1995) Nat. Med. 1:53-8). In addition, implantation of polymer-based bioactive rods that secrete epidermal growth factor and bFGF into transplanted fetal ventral mesencyphalic tissue result in both improved functional characteristics and enhanced cell survival (Tornquvist et al. (2000) Exp. Neurol. 164:130-138).

NGF has also been shown to influence grafted tissue in the CNS. For example, ChAT activity, an assay indicative of cholinergic cell activity, was elevated significantly in cholinergic neurons that were transplanted into brain tissue that contained an NGF-releasing pellet adjacent to the grafted cells (Mahoney et al. (1999) Med. Sci. 96:4536-4539). IGF-I has also been shown to promote differentiation of post-mitotic mammalian CNS neuronal stem cells and to influence apoptosis of human erythroid progenitor cells. See, for example, Arsenijevic et al. (1998) J. Neurosci. 18:2118-2128; Tanigachi et al. (1997) Blood 90:2244-2252; Reboarcet et al. (1996) J. Biol. Reprod. 55:1119-1125; Muta et al. (1994) J. Clin. Invest. 94:34-43; and, Muta et al. (1993) J. Cell. Phys. 156:264-271. Additionally, it has been shown that certain growth associated proteins, such as, GAP-43 and CAP-23 act to promote regeneration of injured axons and may support regeneration in the spinal cord and CNS. See, for example, Bomze et al. (2001) Nature Neurosci. 4:38-43 and Woolf et al. (2001) Nature Neurosci. 4:7-9.

Administration of regulatory agents as a means of improving the clinical outcome of a mammal having undergone a neural regenerative strategy has, however, been meet with difficulty. Generally, these agents cannot be administered systemically. Furthermore, many of these regulatory agents do not cross the blood-brain barrier efficiently. Intracerebroventricular administration, while possibly an effective method for delivering regulatory agents, is an invasive technique that is not preferred in a clinical setting. Implantation of polymers containing regulatory agents is also invasive and is further limited by the relatively small radius surrounding the polymer implant in which the regulatory agent is capable of eliciting an effect. Additionally, while genetic engineering of the transplanted cells to express regulatory agents has been performed, stable transfection and survival of the cells following implantation continues to be problematic. Further, it is likely multiple regulatory factors will be necessary for the successful treatment of the neurodegenerative disorder.

The present invention provides a noninvasive method of delivering a regulatory agent to the CNS, brain, or spinal cord to improve the clinical outcome of a mammal having undergone a neural regenerative strategy comprising the transplantation of a donor cell into the CNS of the mammal.

### SUMMARY OF THE INVENTION

Methods for the treatment and/or prevention of central nervous system disorders are provided. More specifically, the present invention provides a method for regulating the development of a transplanted donor cell in the central nervous system (CNS) of a mammal. The methods of the invention therefore find use in improving the clinical outcome of a mammal having undergone a neural regenerative strategy comprising the transplantation of a donor cell into the CNS of the mammal.

In particular, the present invention provides a method of regulating the development of a donor cell in the CNS of a mammal comprising administering a composition comprising at least one regulatory agent to a tissue of a mammal innervated by the trigeminal nerve, the olfactory nerve, or a combination thereof. The regulatory agent is absorbed through the tissue and transported to the CNS of the mammal in an amount effective to regulate the development of the donor cell. In one embodiment, the method administers the regulatory agent through the mucosa or epithelium of the nasal cavity, tongue, mouth, skin, or conjunctiva.

The present invention further provides a method of administering a regulatory agent for the treatment and/or prevention of a CNS disorder in a mammal having undergone a neural regenerative strategy. In specific embodiments, the central nervous system disorder is a head injury, spinal cord injury, stroke, or ischemia. In one embodiment, the central nervous system disorder is a neurodegenerative disorder and includes epilepsy, Huntington disease, Parkinson's disease, ALS, and Alzheimer's disease. In yet other embodiments, the CNS disorder is a mood disorder. In other embodiments, the method of administering a regulatory agent is used for the treatment and/or prevention of demyelinating or dysmyelinating disorders, including, Pelizaeus-Merzbacher disease and multiple sclerosis.

In specific embodiments of the present invention, the regulatory agent is a growth factor or biologically active variant thereof. In other embodiments, the regulatory agent is IGF-I, bFGF, NGF, or a biologically active variant thereof.

In further embodiments, the regulatory agent modulates the immune response of the subject having undergone the neural regenerative strategy. In specific embodiments, the regulatory agent is an immune suppressant such as a cytokine or cyclosporin.

### DETAILED DESCRIPTION OF THE INVENTION

The present invention is directed to the treatment and/or prevention of CNS disorders. Specifically, the present invention provides a method for regulating the development of a transplanted donor cell in the central nervous system (CNS) of a mammal. The methods comprise administering a therapeutically effective amount of at least one regulatory agent to a tissue innervated by the trigeminal nerve, the olfactory nerve, or a combination thereof to the CNS of the mammal. The methods find use in improving the clinical outcome of a mammal having undergone a neural regenerative strategy comprising the transplantation of a donor cell into the CNS of the mammal.

Administration of at least one regulatory agent by the methods of the present invention will regulate development of the transplanted donor cell. By “regulate development” is intended the regulatory agent potentiates the survival, differentiation, axonal development, dendritic development, and/or proliferation of the transplanted donor cell; improves adhesion of transplanted donor cells to surrounding tissues (i.e., incorporation into parenchymal tissue); improves the capacity of the transplanted donor cell to establish synaptic connection with the host neurons (i.e, enhances nerve fiber formation in the donor cells; increases nerve fiber projection distances of the donor cells; or enhances nerve fiber destiny of the donor cells); and/or instructs the transplanted donor cell to commit to a specific neural lineage (i.e., adopt a neuronal (GABA-ergic neurons, dopaminergic neurons, cholinergic neurons, hippocampal neurons, and the like), astrocytic or oligodendritic cell fate). It is further recognized that a regulatory agent can potentiate the survival of a transplanted donor cell by modulating the immune response of the subject. By “modulate” is intended the down regulation of the immune or inflammatory response (i.e., influencing systemic immune function, antigen presentation, cytokine production, lymphocyte proliferation, and entry of lymphocytes and macrophages into the CNS).

Furthermore, administration of the regulatory agent can “regulate development” of the transplanted donor cell by influencing the developmental cues released by the transplanted donor cells (i.e., promote the donor cell to release neurotransmitters such as, dopamine, acetylcholine, GABA, or other neuroprotective factors). As such, the function and repair (i.e., enhanced nerve fiber formation, nerve fiber projection distances, and/or nerve fiber density) of the surrounding host tissue can be enhanced by the methods of the present invention. Accordingly, the methods find use in enhancing the regeneration or repair of damaged neuronal tissue in a mammal having undergone a neural regenerative strategy that comprises the transplantation of a donor cell into the CNS of the mammal.

The present invention therefore provides a method to improve cell-based therapies used to regenerate neural tissue that has been damaged by any CNS disease or disorder. CNS disorders include, for example, head injury, spinal cord injury, stroke, and ischemia. CNS disorders also include neurodegenerative diseases such as, but not limited to, epilepsy, Huntington disease, Parkinson's disease, ALS, and Alzheimer's disease. Further CNS disorders include CNS damage resulting from infectious diseases such as viral encephalitis, bacterial or viral meningitis and CNS damage from tumors. It is further contemplated that the neural regenerative strategy of the present invention can be used to improve the cell-based replacement therapies used to treat or prevent various demyelinating and dysmyelinating disorders, such as Pelizaeus-Merzbacher disease, multiple sclerosis, various leukodystrophies, post-traumatic demyelination, and cerebrovasuclar accidents. Disorders of the central nervous system further include mental disorders such as mood disorders (i.e., depression, bipolar disorder), anxiety disorders, and schizophrenic disorders. In addition, the present invention may also find use in enhancing the cell-based therapies used to repair damaged spinal cord tissue following a spinal cord injury.

By “treatment or prevention” is intended any enhanced regeneration and/or repair of damaged CNS tissue that occurs following administration of a regulatory agent by the methods of the invention to a mammal having undergone a neural regenerative strategy. The enhanced regeneration and/or repair of damaged CNS tissue includes an improvement in either the rate or the extent of behavioral and/or morphological recovery. Accordingly, the method of the invention “prevents” (i.e., delays or inhibits) and/or “reduces” (i.e., decreases, slows, or ameliorates) the detrimental effects of the CNS disease, injury, or disorder in the mammal receiving the therapy.

Neural regenerative strategies comprising the transplantation of donor cells into the CNS of a host are known in the art. As defined herein, a “donor cell” is a cell that is transplanted into the damaged CNS of a host subject. The donor cell is characterized as being developmentally regulated by the regulatory agent administered by the method of the present invention. A donor cell can be derived from any source and at various stages of developmental differentiation so long as the developmental regulation by the regulatory agent is sufficient to prevent or reduce the morphological and/or behavioral neurological symptoms of the disorder being treated. It is recognized that the donor cell can be either heterologous or analogous to the host. By heterologous is intended the donor cell is derived from a mammal other than the host, while an analogous donor cell is derived from the host, manipulated ex vivo, and transplanted back into the host subject.

The donor cell of the invention can be derived from any fetal or adult neural tissue, including tissue from the hippocampus, cerebellum, spinal cord, cortex (i.e., motor or somatosensory cortex), striatum, basal forebrain (cholenergic neurons), ventral mesencephalon (cells of the substantia nigra), and the locus ceruleus (neuroadrenaline cells of the central nervous system).

As used herein, the terms “differentiate” and “mature” refer to the progression of a cell from a stage of having the potential to differentiate into at least two different cellular lineages to becoming a specialized cell. Such terms can be used interchangeably for the purposes of the present invention. The term “lineage” refers to all of the stages of the developmental cell type, from the earliest precursor cell to a completely mature cell (i.e., a specialized cell). Accordingly, the transplanted donor cells can be derived from a multipotent cell lineage, preferably a neuronal lineage, and may be in any stage of differentiation.

Multipotent stem cells are characterized by their ability to undergo continuous cellular proliferation, to regenerate exact copies of themselves (self-renewal), to generate a large number of regional cellular progeny, and to elaborate new cells in response to injury or disease. As used herein, the term “multipotent stem cell” refers to a cell capable of differentiating into a variety of lineages. Preferably a multipotent stem cell is able to differentiate into at least two cell lineages. A “multipotent population of cells” refers to a composition of cells capable of differentiating into less than all lineages of cells but at least into two cell lineages.

A “neural stem cell” is defined herein as a multipotent cell that is an immature and uncommitted multipotent cell that exists in the nervous system (Ourednik et al. (1999) Clinical Genetics 56:267-278). Under specific conditions, the neural stem cell is capable of producing daughter cells that can terminally differentiate into neurons and glia (i.e., astrocytes (type I and II) and oligodendrocytes). They exist in both the developing nervous system and in the adult nervous system. A detailed characterization of the properties of neural stem cells can be found in, for example, McInnes et al. (1999) Clin. Genet. 56:267-278, herein incorporated by reference. Current studies have demonstrated that multipotent stem cells from a non-neurologic region are not lineage-restricted to their developmental origin, but can generate region-specific neurons when exposed to the appropriate environmental cues (Lamga et al. (2001) J. Neurosci. 20:8727-8735).

A “neuronal progenitor cell” is an undifferentiated cell that is derived from a neural stem cell and which has committed to a particular path of differentiation, does not exhibit self-maintenance, and under appropriate conditions will differentiate into neuroblasts (neuron generating cells) or fibroblasts (glia generating cells). The use of such multipotent neuronal cell lineages for transplantation is known in the art. See, for example, Snyder et al.(1992) Cell 68:33, where multipotent neuronal cell lines have been grafted into the rat cerebellum to form neurons and glial cells. See, also, Campell et al. (1995) Neuron 15:1259-1273; Fishell et al. (1995) Development 121:803-812; and, Olsson et al. (1995) Eur. J. Neurosci. 10:71-85.

The developing and the adult mammalian central nervous system contains a population of neural stem cells and progenitor cells that are of particular interest in the present invention. Methods of isolation and transplantation of various neural progenitor cells derived from different tissues at different developmental stages are known in the art and include, for example, striatum cortex (Winkler et al. (1998) Mol. Cell. Neurosci. 11:99-116; Hammang et al. (1997) Exp. Neurol. 147:84-95); cortex (Brustle et al. (1998) Nat. Biotechnol 16:1040-1044 and Sabate et al. (1995) Nat. Genet 9:256-260); human telencephalon (Flax et al (1998) Nature 392:18-24 and Vescovi et al. (1999) Neuron 11:951-966); hippocampus (Gage et al. (1995) J. Neurobiol. 36:249-266 and Suhonen et al. (1996) Nature 383:624-627); basal forebrain (Minger et al. (1996) Exp. Neurol. 141:12-24); ventral mesencephalon (Winkler et al. (1998) Mol. Cell. Neurosci. 11:99-116; Svendsen et al. (1996) Exp. Neurol 137:376-388; Hammang et al. (1997) Exp. Neurol. 147:84-95; Studer et al. (1997) Nat. Neurosci. 1:290-295; Milward et al. (1997) J. Neurosci. Res. 50:862-871); and subventricular zone (Milward et al. (1997) Milward et al. (1997) J. Neurosci. Res. 50:862-871). Each of these references is herein incorporated by reference. In addition, methods for the isolation of neural stem cell progeny and method to promote their differentiation can also be found in U.S. Pat. No. 6,071,889 and U.S. Pat. No. 6,103,530, both of which are herein incorporated by reference. Methods for the isolation and culturing of neuroblasts are provided in U.S. Pat. No. 6,045,807, herein incorporated by reference.

Donor cells can also be of paraneural origin. A preferred example of such a cell is the adrenal medullar chromafin cell. See, for example, Bjorklund et al. (1985) Neural Grafting in the Mammalian CNS (Amsterdam: Elsevier), pp. 3-11, and Lindvall et al (1997) Ann. Neurol 22: 457-468, which demonstrate the usefulness of chromafin cells for the treatment of Parkinson's disease.

Donor cells that are not of neural origin, but which have been altered to produce a substance of neurological interest, can also be used. A preferred cell type is a human foreskin fibroblast, which is easily obtained and cultured (see, for example, U.S. Pat. No. 6,060,048). Such cells are preferably genetically altered, using methods known in the art, to express neuronal growth factors, neurotransmitters, neuropeptides, or enzymes involved in brain metabolism. See, for example, Gage et al. (1987) Neurosci. 23: 795-807; Rosenberg et al. (1988) Science 242: 1575-1578; Shimohama et al. (1989) Mol. Brain Res. 5: 271-278; which are hereby incorporated by reference. Alternatively, donor cells derived from a non-neuronal origin, such as epidermal cells, may be converted or transdifferentiated into different types of neuronal cells. See, for example, U.S. Pat. No. 6,087,168.

The donor cell of the present invention may be genetically altered prior to transplantation into the host. As used herein, the term “genetically altered” refers to a cell into which a foreign nucleic acid, e.g., DNA, has been introduced. The foreign nucleic acid may be introduced by a variety of techniques, including, but not limited to, calcium-phosphate-mediated transfection, DEAE-mediated transfection, microinjection, viral transformation, protoplast fusion, and lipofection. The genetically altered cell may express the foreign nucleic acid in either a transient or long-term manner. In general, transient expression occurs when foreign DNA does not stably integrate into the chromosomal DNA of the transfected cell. In contrast, long-term expression of foreign DNA occurs when the foreign DNA has been stably integrated into the chromosomal DNA of the transfected cell.

Such genes of interest include neurotransmitter-synthesizing enzymes (i.e., tyrosine hydrolase (TH) and cholineacetyltransferase). Such methods are commonly known in the art. For instance, donor cells from various regions of the brain and at different stages of development have been isolated and have been immortalized via genetic alteration. For example, olfactory and cerebellum cells have been immortalized using the viral myc (v-myc) oncogene to generate cell lines with neuronal and glial phenotypes (Ryder et al. (1990) J. Neurobiol. 21:356). Similar studies by Snyder et al. ((1992) Cell 68:33) resulted in multipotent neuronal cell lines that were engrafted into the rat cerebellum to form neurons and glial cells. In other studies, murine neuroepithelial cells were immortalized with a retrovirus vector containing c-myc and were cultured with growth factors to form differentiated cell types similar to astrocytes and neurons (Barlett et al. (1988) Proc. Natl. Acad. Sci. USA 85:3255).

Regulatory Agents

As used herein, “regulatory agent” refers to any molecule having a growth, proliferative, differentiative, or trophic effect on a transplanted donor cell of the present invention. Any regulatory agent that is capable of regulating the development of the transplanted donor cell can be administered by the methods of the present invention. See, for example, Mackay-Sim et al. (2000) Prog. Neurobiol. 62:527-559, herein incorporated by reference.

Regulatory agents of interest include, for example, agents that promote the survival of the donor cells by modulating the immune and inflammatory response. Such regulatory agents include, for example, cyclosporin and various other immunomodulators, including, interleukins (i.e., IL-1, IL-2, IL-3, IL-4, IL-5, IL-6, IL-7, IL-8, IL-9, IL-10); tumor necrosis factors (i.e., TNF-α and TNF-β); and, interferons (i.e., IFN-α, IFN-β, IFN-γ, IFN-ω, and IFN-τ); and any biologically active variants thereof. Further details regarding the administration of these immunomodulating agents by the methods of the present invention can be found in U.S. patent Ser. No. 09/733,168, entitled “Methods for Administering a Cytokine to the Central Nervous System and the Lymphatic System,” filed on Dec. 9, 2000, herein incorporated by reference.

Additional regulatory agents that find use in the methods of the invention include CAP23, a major cortical cytoskeleton-associated and calmodulin binding protein, and GAP43, a neural growth-associated protein. See, for example, Frey et al. (2000) J. Cell. Biol. 7:1443-1453. Further agents of interest include Osteogenic Protein-1 (OP-1) which is a morphogenic protein that stimulates growth, differention, and differentiation maintenance (U.S. Pat. No. 6,153,583); sonic hedgehog, a polypeptide shown to promote the survival of dopaminergic neurons (Miao et al. (1996) Cell Transplant 55:2-17); various other glial growth factors (U.S. Pat. Nos. 5,716,930; 6,147,190; and 5,530,109); and any biologically active variants thereof. All of these references are herein incorporated by reference.

Other regulatory agents of interest include growth factors. As used herein “growth factor” refers to a polypeptide capable of regulating the development of the transplanted donor cell. Growth factors useful in the methods of the present invention include, but are not limited to, members of the neurotrophin family (i.e., nerve growth factor (NGF), brain-derived neurotrophic factor (BDNF), neurotrophin-3 (NT-3), and neurotrophin-4 (NT-4, also known as NT-4/5 or NT-5); fibroblast growth factors (FGFs, i.e., basic fibroblast growth factor); epidermal growth factor family (i.e., EGF, TGFα, amphiregulin, heparin-binding EGF-like growth factor (HB-EGF), batacelluin (BTC), and the neuregulin group); platelet-derived growth factor; insulin; insulin-like growth factors (i.e., IGF-I and IGF-2); ciliary neurotrophic factor (CNTF), glia cell line-derived neurotrophic factor family (GDNF) (i.e., GDNF and neurturin (NTN), persephin (PSP), and artemin (ART)); transforming growth factor β superfamily (i.e., subfamilies include TGFβ1, TGFβ2, TGFβ3, TGFβ4, TGFβ5, activin, inhibin, decapentaplegic); growth differentiation factors (GDF) (i.e., GDF1, GDF2, GDF3, GDF5, GDF6, GDF7, GDF8, GDF9, GDF9B, GDF10, GDF11, and GDF15); glia-derived nexin; activity dependent neurotrophic factor (ADNF); glial growth factor (GGF); and the like. It is further recognized that any biologically active variant of these growth factors is also useful in the methods of the present invention.

The regulatory agent to be administered in the methods of the present invention can be from any animal species including, but not limited to, rodent, avian, canine, bovine, porcine, equine, and, preferably, human. Preferably the regulatory agent administered is from the same species as the mammal undergoing treatment.

Biologically active variants of regulatory polypeptides (i.e., growth factors, such as IGF-I, NGF, and basic FGF, cytokines, etc.) are also encompassed by the methods of the present invention. Such variants should retain the biological activity of the regulatory agent, particularly the ability to regulate the development of the donor cell (i.e., promote the survival, maintain the desired phenotype, and/or regulate the developmental cues produced by the donor cell). For example, when the regulatory polypeptide is a growth factor, such as IGF-I, NGF-I, or a member of the FGF family, the ability to bind their respective receptor sites will be retained. Such receptor binding activity may be measured using standard bioassays. Representative assays include known radioreceptor assays using placental membranes (see, e.g., U.S. Pat. No. 5,324,639; Hall et al. (1974) J. Clin. Endocrinol. Metab. 39:973-976; and Marshall et al. (1974) J. Clin. Endocrinol. Metab. 39:283-292), a bioassay that measures the ability of the molecule to enhance incorporation of tritiated thymidine, in a dose-dependent manner, into the DNA of BALB/c 3T3 fibroblasts (see, e.g., Tamura et al. (1989) J. Biol. Chem. 262:5616-5621), and the like; herein incorporated by reference. Preferably, the variant has at least the same activity as the native molecule.

Suitable biologically active variants can be fragments, analogues, and derivatives of the regulatory agent (i.e., growth factor polypeptides, such as IGF-I, NGF, and basic FGF, cytokines, etc.). By “fragment” is intended a protein consisting of only a part of the intact regulatory polypeptide sequence. The fragment can be a C-terminal deletion or N-terminal deletion of the regulatory polypeptide. By “analogue” of a regulatory agent (ie., growth factors, such as IGF-I, NGF, and bFGF, cytokines, etc.) is intended an analogue of either the full length polypeptide capable of regulating the development of the donor cell, or a fragment thereof, that includes a native sequence and structure having one or more amino acid substitutions, insertions, or deletions. Peptides having one or more peptoids (peptide mimics) are also encompassed by the term analogue (see i.e., International Publication No. WO 91/04282). By “derivative” of a regulatory agent (i.e., growth factors, such as IGF-I, NGF, and bFGF, cytokines, etc.) is intended any suitable modification of the native polypeptide or fragments thereof, or their respective analogues, such as glycosylation, phosphorylation, or other addition of foreign moieties, so long as the activity is retained.

Preferably, naturally or non-naturally occurring variants of a regulatory polypeptide have amino acid sequences that are at least 70%, preferably 80%, more preferably, 85%, 90%, 91%, 92%, 93%, 94% or 95% identical to the amino acid sequence to the reference molecule, for example, a regulatory agent such as a native human growth factor (i.e., IGF-I, NGF, and bFGF) or a native human cytokine, or to a shorter portion of the reference regulatory agent (i.e., a growth factor or cytokine molecule). More preferably, the molecules are 96%, 97%, 98% or 99% identical. Percent sequence identity is determined using the Smith-Waterman homology search algorithm using an affine gap search with a gap open penalty of 12 and a gap extension penalty of 2, BLOSUM matrix of 62. The Smith-Waterman homology search algorithm is taught in Smith and Waterman (1981) Adv. Appl. Math. 2:482-489. A variant may, for example, differ by as few as 1 to 10 amino acid residues, such as 6-10, as few as 5, as few as 4, 3, 2, or even 1 amino aid residue.

With respect to optimal alignment of two amino acid sequences, the contiguous segment of the variant amino acid sequence may have additional amino acid residues or deleted amino acid residues with respect to the reference amino acid sequence. The contiguous segment used for comparison to the reference amino acid sequence will include at least 20 contiguous amino acid residues, and may be 30, 40, 50, or more amino acid residues. Corrections for sequence identity associated with conservative residue substitutions or gaps can be made (see Smith-Waterman homology search algorithm).

The art provides substantial guidance regarding the preparation and use of such variants, as discussed further below. A fragment of a regulatory polypeptide will generally include at least about 10 contiguous amino acid residues of the full-length molecule, preferably about 15-25 contiguous amino acid residues of the full-length molecule, and most preferably about 20-50 or more contiguous amino acid residues of full-length regulatory polypeptide.

Insulin-like Growth Factor-I (IGF-I):

A growth factor useful in the present invention is IGF-I. The term “IGF-I” as used herein refers to insulin-like growth factor I (IGF-I), a single-chain peptide having 70 amino acids and a molecular weight of about 7,600 daltons. Insulin-like growth factor I stimulates mitosis and growth processes associated with cell development. The amino acid and nucleotide sequence for IGF-I is known in the art. See, for example, U.S. Pat. No. 5,324,639 which discloses the human IGF-I sequence; Genbank Accession No. X15726, which discloses the sequence of bovine IGF-I; and Genbank Accession No. X06043 which discloses the sequence of rat IGF-I. Each of these references is herein incorporated by reference.

In one embodiment of the present invention, the regulatory agent is IGF-I or a biologically active variant thereof. Several biologically active variants (i.e., analogues, fragments, and derivatives) of IGF-I are known in the art and include those described in, for example, Proc. Natl. Acad. Sci. USA 83 (1986):4904-4907; Biochem. Biophys. Res. Commun. 149 (1987):398-404; J. Biol. Chem. 263 (1988):6233-6239; Biochem. Biophys. Res. Commun. 165 (1989):766-771; Forsbert et al. (1990) Biochem. J. 271:357-363; U.S. Pat. Nos. 4,876,242 and 5,077,276; and International Publication Nos. WO 87/01038 and WO 89/05822. Representative analogues include one with a deletion of Glu-3 of the mature molecule, analogues with up to 5 amino acids truncated from the N-terminus, an analogue with a truncation of the first 3 N-terminal amino acids (referred to as des(1-3)-IGF-I, des-IGF-I, tIGF-I, or brain IGF), and an analogue including the first 17 amino acids of the B chain of human insulin in place of the first 16 amino acids of human IGF-I.

The IGF-I used in the present invention can be in its substantially purified, native, recombinantly produced, or chemically synthesized forms. For example, the IGF-I can be isolated directly from blood, such as from serum or plasma, by known methods. See, for example, Phillips (1980) New Eng. J. Med. 302:371-380; Svoboda et al. (1980) Biochemistry 19:790-797; Cornell and Boughdady (1982) Prep. Biochem. 12:57; Cornell and Boughdady (1984) Prep. Biochem. 14:123; European Patent No. EP 123,228; and U.S. Pat. No. 4,769,361. IGF-I may also be recombinantly produced in the yeast strain Pichia pastoris and purified essentially as described in U.S. Pat. Nos. 5,324,639, 5,324,660, and 5,650,496 and International Publication No. WO 96/40776. Alternatively, IGF-I can be synthesized chemically, by any of several techniques that are known to those skilled in the peptide art. See, for example, Li et al. (1983) Proc. Natl. Acad. Sci. USA 80:2216-2220, Stewart and Young (1984) Solid Phase Peptide Synthesis (Pierce Chemical Company, Rockford, Ill.), and Barany and Merrifield (1980) The Peptides: Analysis, Synthesis, Biology, ed. Gross and Meienhofer, Vol. 2 (Academic Press, New York, 1980), pp. 3-254, for solid phase peptide synthesis techniques; and Bodansky (1984) Principles of Peptide Synthesis (Springer-Verlag, Berlin); and Gross and Meienhofer, eds. (1980) The Peptides: Analysis, Synthesis, Biology, Vol. 1 (Academic Press, New York), for classical solution synthesis. IGF-I can also be chemically prepared by the method of simultaneous multiple peptide synthesis. See, for example, Houghten (1985) Proc. Natl. Acad. Sci. USA 82:5131-5135; and U.S. Pat. No. 4,631,211. These references are herein incorporated by reference. Furthermore, methods to prepare a highly concentrated, low salt-containing, biologically active form of IGF-I or variant thereof are provided in International Publication No. WO 99/24062, entitled “Novel IGF-I Composition and Its Use”; herein incorporated by reference. Further methods for making IGF-I fragments, analogues, and derivatives are available in the art. See generally U.S. Pat. Nos. 4,738,921, 5,158,875, and 5,077,276; International Publication Nos. WO 85/00831, WO 92/04363, WO 87/01038, and WO 89/05822; and European Patent Nos. EP 135094, EP 123228, and EP 128733; herein incorporated by reference.

Thus, the IGF-I administered may be derived from any method known in the art. In one embodiment, the IGF-I administered is derived from a viscous syrup as described in WO 99/24062. Aliquots of this highly concentrated IGF-I syrup may be reconstituted into an injectable or infusible form such as a solution, suspension, or emulsion. It may also be in the form of a lyophilized powder, which can be converted into a solution, suspension, or emulsion before administration.

When administering IGF-I, appropriate serum glucose monitoring should be done to prevent hypoglycemia. The half-times of elimination, volumes of distribution, daily production rates, and serum concentrations are well established pharmacokinetic parameters for IGF-I in normal humans. See, for example, Guler et al. (1989) Acta Endocrinological (Copenh) 121:753-58); Zapf et al. (1981) J. Clin. Invest. 68:1321-30.

Fibroblast Growth Factors (FGF):

In another embodiment of the present invention, the regulatory agent is a member of the FGF family of growth factors and/or biologically active variants thereof. The fibroblast growth factor family encompasses a group of structurally related proteins that bind heparin with a high affinity. FGF family members have mitogen activity and induce the proliferation of a wide variety of cell types. FGF family members also participate in angiogenesis, differentiation, cell migration, embryo development, and neuronal maintenance/survival. The term “FGF” as used herein refers to a member of the fibroblast growth factor family including, for example, FGF-1 (acidic FGF), FGF-2 (basic FGF), FGF-3, FGF-4, FGF-5, FGF-6, FGF-8, FGF-9, FGF-98, or a biologically active fragment or variant thereof. The amino acid sequence and methods for making many of the FGF family members are well known in the art. In particular, references disclosing the amino acid sequence and recombinant expression of various FGF family members include, for example, human and bovine FGF-1, U.S. Pat. No. 5,604,293; human and bovine FGF-2, U.S. Pat. No. 5,439,818 and U.S. Pat. No. 5,155,214, respectively; bovine and murine FGF-3, Dickson et al. (1987) Nature 326:833; human FGF-4 Yoshida et al. (1987) Proc. Natl. Acad. Sci. USA 84:7305-7309; human FGF-5, Zhan et al. (1988) Mol. Cell. Biol. 8(8):3487-3495; Human FGF-6, Coulier et al. (1991) Oncogene 6:1437-1444; human FGF-7 (KGF), Miyamoto et al. (1993) Mol. Cell. Biol. 13(7):4251-4259; murine FGF-8, Tanaka et al. (1992) Proc. Natl. Acad. Sci. USA 89:8928-8932; human and marine FGF-9, Santos-Ocamp et al. (1996) J. Biol. Chem. 271(3):1726-1731 (1996); and human FGF-18 (FGF-98), U.S. Provisional Patent Application Serial No. 60/083,553. Each of these references is herein incorporated by reference.

Biologically active variants of FGF (i.e., analogues, derivatives, and fragments) should retain FGF activities, particularly the ability to regulate the development of the donor cell. FGF activity can be measured using standard bioassays, such as those described above. Further assays include measuring the binding affinity of FGF family members to FGF receptors and/or heparin (Moscatelli et al. (1987) J. Cell Physiol. 131:123-30), determining FGF mitogenic activities (U.S. Pat. No. 5,310,883; U.S. Pat. No. 6,071,889; and Ray et al. (1997) Proc. Natl. Acad. Sci. 94:7047-7052), and the like. All of these references are herein incorporated by reference.

In specific embodiments of the invention, the preferred FGF family member is bFGF (FGF-2). Several variants (i.e., analogues, derivatives, and fragments) of bFGF are described in, for example, U.S. Pat. No. 5,851,990, Zhu et al. (1991) Science 251:90-93, Biochem. Biophys. Comm. 151 (1988):701-708, EP No. 281,822, EP No. 326,951, EP No. 298,728, EP No. 320,148, EP No. 319,052, EP No. 298,723, EP No. 363,675, WO 89/04832, and U.S. Pat. No. 5,310,883; all of which are herein incorporated by reference.

The FGF for the present invention may be derived from various mammalian tissues known to express the factor of interest. Basic FGF, for example, may be extracted from the brain and pituitary. FGF purification can be achieved by heparin-sepharose column chromatography as described in Gospodarowicz et al. (1984) Proc. Natl. Acad. Sci. USA 81:6963-6967 or in U.S. Pat. No. 5,310,883. Purification may also be achieved using β-cytodextin tetradeca sulfate affinity chromatography as described in Shing et al. (1990) Anal. Biochem. 185:108-111; all of which are herein incorporated by reference. Recombinant bFGF can be made as described in U.S. Pat. No. 5,155,214, entitled “Basic Fibroblast Growth Factor,” herein incorporated by reference. Methods of purifying recombinant bFGF to pharmaceutical quality can be found in, for example, U.S. Pat. No. 4,956,455.

Nerve Growth Factor (NGF)

In another embodiment of the present invention, the regulatory agent is nerve growth factor (NGF) or a biologically active variant thereof. NGF was originally isolated as a complex having a molecular weight of 130 kDa and a sedimentation coefficient of 7S. This 7S complex included three types of subunits, with the “β” subunit carrying all of the biological activities of NGF. Nerve growth factor stimulates mitosis and growth processes of cells, particularly nerve cells, and regulates development (i.e., influences repair, survival, and differentiation). The preferred amino acid sequence for human pre-pro-NGF and human mature NGF are provided in U.S. Pat. No. 5,288,622, which is incorporated herein by reference.

Biologically active variants (i.e., analogues, derivatives, and fragments thereof) of NGF are also encompassed by the method of the present invention. Such variants should retain NGF activities, particularly the ability to regulate the development of donor cells and/or the ability to interact with the Trk family of tyrosine kinase receptors. These activities may be measured using the methods described herein. Furthermore, representative assays include known radioreceptor assays using placental membranes (see, for example, U.S. Pat. No. 5,324,639; Hall et al. (1974) J. Clin. Endocrinol. Metab. 39:973-976; and Marshall et al. (1974) J. Clin. Endocrinol. Metab. 39:283-292), and promoting survival of chick embryo sympathetic ganglion neurons (Greene et al. (1977) Develop. Biol. 58:96-113 and U.S. Pat. No. 5,986,070). The biologically active variant has at least the same activity as the native molecule.

The NGF used in the present invention can be in its substantially purified, native, recombinantly produced form or in a chemically synthesized form. For example, the NGF can be isolated directly from cells naturally expressing NGF. NGF may also be recombinantly produced in eukaryotic or prokaryotic cell expression systems as described in Edwards et al. (1988) Mol. Cell. Biol. 8:2456; U.S. Pat. No. 5,986,070; and U.S. Pat. No. 6,005,081; all of which are herein incorporated by reference. In such systems, the full-length NGF precursor is first synthesized and then proteolytically processed to produce mature NGF that is correctly folded 3-dimensionally. Such a method of refolding is described in, for example, U.S. Pat. No. 5,986,070. Alternatively, NGF can be synthesized chemically, by any of several techniques that are known to those skilled in the peptide art. See, for example, Li et al. (1983) Proc. Natl. Acad. Sci. USA 80:2216-2220, Stewart et al. (1984) Solid Phase Peptide Synthesis (Pierce Chemical Company, Rockford, Ill.), and Barany et al. (1980) The Peptides: Analysis, Synthesis, Biology, ed. Gross and Meienhofer, Vol. 2 (Academic Press, New York, 1980), pp. 3-254, for solid phase peptide synthesis techniques; and Bodansky (1984) Principles of Peptide Synthesis (Springer-Verlag, Berlin); and Gross et al. (1980) The Peptides: Analysis, Synthesis, Biology, Vol. 1 (Academic Press, New York), for classical solution synthesis. NGF can also be chemically prepared by the method of simultaneous multiple peptide synthesis. See, for example, Houghten (1985) Proc. Natl. Acad. Sci. USA 82:5131-5135; and U.S. Pat. No. 4,631,211. These references are herein incorporated by reference. Furthermore, control release formulations and stabilizing formulations of NGF can be found in, for example, U.S. Pat. Nos. 6,113,947 and 6,090,781, respectively.

Methods of Administering the Regulatory Agent

The methods of the present invention comprise regulating the development of a transplanted donor cell in the CNS of a mammal by administering a therapeutically effective amount of at least one regulatory agent to a tissue innervated by the trigeminal nerve and/or the olfactory nerve. Administration of the regulatory agent via the trigeminal or olfactory nerve allows for the transport of the agent to a variety of CNS structures including, for example, the olfactory bulbs; the anterior olfactory nucleus; the midbrain; the medulla; the pons; the cerebellum; the hippocampal formation; the diencephalon; the frontal, temporal, occipital, and parietal cortices; the cervical spinal cord; the brain stem; the basal forebrain; and the caudate/putamen.

The method of administering the regulatory agent via the trigeminal and/or olfactory nerve allows for a more effective delivery of this regulatory agent to the CNS, brain, and/or spinal cord, decreases the amount of this agent administered outside the CNS, brain, and/or spinal cord, and, preferably decreases the potential undesirable systemic effects of this agent. Accordingly, the more effective and/or efficient delivery of the regulatory agent to the CNS, brain, and/or spinal cord may allow the total dose of regulatory agent that must be administered to regulate development of a transplanted donor cell to be decreased.

Methods for delivering various agents to the CNS via the trigeminal nerve and/or the olfactory nerve can be found in, for example, WO 00/33813; U.S. patent application Ser. No. 08/780,355; WO 00/33814; and copending U.S. patent application Ser. No. 09/733,168, entitled “Method for Administering a Cytokine to the Central Nervous System and Lymphatic System,” filed on Dec. 9, 2000; all of which are herein incorporated by reference.

Delivery of a therapeutically effective amount of a regulatory agent to a transplanted donor cell located in the CNS, brain, and/or spinal cord of a mammal may be obtained via administration of a pharmaceutical composition comprising a therapeutically effective dose of this agent. By “therapeutically effective amount” or “dose” is meant the concentration of regulatory agent that is sufficient to elicit the desired therapeutic effect with respect to regulating the development of a donor cell, as described herein. Accordingly, an effective amount of the regulatory agent augments the clinical outcome of the cell replacement therapy in comparison to animals treated with only the cell replacement strategy. As such, a therapeutically effective dose can be assayed via a reduction in neural deficits associated with the CNS disorder being treated, and hence is characterized by an improvement in clinical symptoms.

Methods to quantify the extent of neurologic damage and to determine if the CNS disorder has been treated are well known to those skilled in the art. Such methods include, but are not limited to, histological methods, molecular marker assays, and functional/behavior analysis. For example, enhanced functional integration of the donor cells and/or enhanced function and repair of the surrounding neuronal tissue can be assayed by examining the restoration of various functions including cognitive, sensory, motor, and endocrine. Motor tests include those that quantitate rotational movement away from the degenerative side of the brain, and those that assay for balance, coordination, slowness of movement, rigidity, and tremors. Cognitive tests include memory tests and spatial learning. The specific assays used to determine treatment of a neurologic disease will vary depending on the disorder.

Desired biological activities beneficial to the regulation of donor cell development include, for example, potentiation of the survival and/or proliferation of the transplanted donor cell; improvement in the capacity of the transplanted donor cell to establish synaptic connection with the host neurons; and/or instruction of the transplanted donor cell to commit to a specific neural lineage. Methods to assay such events are known in the art. For example, an improvement in the survival of the transplanted donor cells following the administration of the regulatory agent can be assayed using various non-invasive scans such as computerized axial tomography (CAT scan or CT scan), nuclear magnetic resonance or magnet resonance imaging (NMR or MRS) or positron emission tomography (PET) scans. Alternatively, donor cell survival can be assayed post-mortem by microscopic examination of the region of donor cell transplantation. The region of donor cells can be identified, for example, by assaying for molecular markers specific to the donor cells or alternatively, by prior incorporation of tracer dyes. Such dyes include, for example, rhodamine- or flourescein-labeled microspheres, fast blue, or retrovirally introduced histochemical markers.

The therapeutically effective amount will depend on many factors including, for example, the CNS disorder being treated, the type of donor cell transplanted into the mammal, and the responsiveness of the subject undergoing treatment. It is further recognized that the therapeutically effective amount will depend on the type of developmental regulation of the donor cell that is desired (i.e., potentiation of the survival and/or proliferation of the transplanted donor cell; improvement of the capacity of the transplanted donor cell to establish synaptic connection with the host neurons; regulation of the developmental cues released by the transplanted donor cells; or improved function and repair of the surrounding neural tissue). Methods to determine efficacy and dosage are known to those skilled in the art.

For example, in Parkinson's disease, the neurons that degenerate are the dopaminergic neurons of the substantia nigra. Cell replacement strategies for patients with advanced Parkinson's disease are known and include, for example, intrastriatal grafts of nigral dopaminergic neurons from 6- to 9-week-old human embryos (Olanow et al. (1996) Trends Neurosci. 19:102-109 and Lindvall et al. (1999) Mov. Disord. 14:201-205). Delivery of pharmacologically active regulatory agents to regions of the brain effected by Parkinson's disease (i.e., midbrain and substantia nigra) has been demonstrated. See, for example, International Publication No. WO 00/33813 and U.S. patent application Ser. No. 09/733,168, entitled “Methods for Administering a Cytokine to the Central Nervous System and the Lymphatic System,” filed on Dec. 9, 2000; both of which are herein incorporated by reference.

As used herein, an “effective amount” of a regulatory agent for the treatment of Parkinson's disease using the administration method of the present invention will be sufficient to reduce or lessen the clinical symptoms of Parkinson's disease. As such, an effective amount of the regulatory agent (i.e., growth factor) administered by the methods of the present invention will augment the cell replacement strategies performed in the art for the treatment of Parkinson's disease. Accordingly, the methods of the invention enhance survival and/or improve clinical status of the treated animals in comparison to animals treated with cell replacement strategy alone. Improvement in clinical status for Parkinson's disease includes, for example, improvement in the ventral mesencephalic graft efficacy in terms of apomorphine-induced rotational decrease, an increase in the density of striatal reinnervation, and an enhancement in neuronal survival (Tornqvist et al. (2000) Exp. Neurol. 164:130-138). Specific assays for these clinical improvements include using positron emission tomography (PET); normalization of dopamine synthesis and storage as assessed by striatal ¹⁸fluorodopa uptake; and spontaneous and drug-induced dopamine release as measured as dopamine D₂ receptor occupancy in the grafted putamen. See, for example, Piccini et al. (1999) Nat. Neurosci. 2:1137-1140, herein incorporated by reference. Such assays can be readily used by one skilled in the art to determine the dosage range and/or appropriate regulatory agent of choice for the effective treatment of Parkinson's disease.

Huntington disease is characterized by progressive neurodegeneration, particularly in the striatum and cortex, which induces severe impairments in both motor and cognitive functions. Current cell replacement therapies replace inhibitor connections from the striatum to other structures such as the globus pallidus through the implantation of striatal precursor cells. Delivery of pharmacologically active regulatory agents to regions of the brain that are effected by Huntington disease (i.e., caudate-putamen, thalamus, dincephalon, cerebellum, and frontal cortex) has been demonstrated. See, for example, International Publication No. WO 00/33813 and U.S. patent application Ser. No. 09/733,168, entitled “Methods for Administering a Cytokine to the Central Nervous System and the Lymphatic System,” filed on Dec. 9, 2000; both of which are herein incorporated by reference.

As used herein, an “effective amount” of a regulatory agent for the treatment of Huntington disease using the administration method of the present invention will be sufficient to reduce or lessen the clinical symptoms of Huntington disease. Thus, an effective amount of the regulatory agent (i.e., growth factor) administered by the methods of the present invention will augment the cell replacement strategies commonly performed in the art for the treatment of Huntington disease. As such, the methods of the invention enhance survival and/or improve clinical status of the treated animals in comparison to animals treated with cell replacement strategy alone. Improvement in clinical status includes, for example, disinhibition of pallidal output, reduced locomotor hyperactivity, recovery of complex motor and cognitive behavior, and restitution of new habit-learning systems in the lesioned striatum. See, for example, Bjorklund et al. (1994) Functional Neural Transplantation (Raven, N.Y.), pp. 157-195; Dunnett et al. (1995) Behav. Brain Res. 66:133-142; Kendall et al. (1998) Nat. Med. 4:727-729; Palfi et al. (1998) Nat. Med. 4:963-966; Brasted et al. (1999) Proc. Natl. Acad. Sci. USA 96:10524-10529; and Wictorin et al. (1992) Prog. Neurobiol. 38:611-639; all of which are herein incorporated by reference. Administration of regulatory agents by the methods of the present invention will be sufficient to improve the clinical outcome of the cell replacement therapy. Such assays can be readily used by one skilled in the art to determine the dosage range and/or appropriate regulatory agent of choice for the effective treatment of Huntington disease.

Ischemic damage to the CNS, brain, or spinal cord can result from, for example, cardiac arrest or coronary artery occlusion, or cerebral artery occlusion or stroke. Neural circuits of the CNS damaged following an ischemic event have been reconstructed using various cell replacement strategies. For instance, for focal ischemia events, implantation of embryonic striatum into the damaged striatum (Hodges et al. (1994) Functional Neural Transplantation (Raven, N.Y.), pp. 347-386) and implantation of neurons derived from a human teratocarcinoma cell line (Borlongan et al. (1998) Exp. Neurol. 149:310-321 and Borlongan et al.(1998) Neuroreport 9:3703-3709) have been performed. See also, for example, Hodges et al. (1996) Neurosci. 72:959-988, Sorensen et al. (1996) Exp. Neurol. 138:227-235, and Sinden et al. (1997) Neurosci. 81:599-608.

As used herein, an “effective amount” of a regulatory agent for the treatment of ischemic injury will be sufficient to reduce or lessen the clinical symptoms of the ischemic event. As such, an effective amount of the regulatory agent (i.e., growth factor) administered by the methods of the present invention will augment the cell replacement strategies commonly performed in the art for the treatment of an ischemic injury. Improvement in clinical status includes, for example, a reduction in infarct size, edema, and/or neurologic deficits (i.e., improved recovery of motor, sensory, vestibulomoter, and/or somatosensory function). Improvements further encompass a reduction in neural deficits, and hence improved recovery of motor, sensory, vestibulomoter, and/or somatosensory function.

Methods to determine if an ischemic event has been treated, particularly with regard to reduction of ischemic damage including infarct size, edema, and development of neural deficits, are well known to those skilled in the art. For example, after ischemic injury, there is a significant increase in the density of omega 3 (peripheral-type benzodiazepine) binding sites (Benazodes et al. (1990) Brain Res. 522:275-289). Methods to detect omega 3 sites are known and can be used to determine the extent of ischemic damage. See for example, Gotti et al. (1990) Brain Res. 522:290-307 and references cited therein. Alternatively, Growth Associated Protein-43 (GAP-43) can be used as a marker for new axonal growth following an ischemic event. See, for example, Stroemer et al. (1995) Stroke 26:2135-2144, and Vaudano et al. (1995) J. Neurosci 15:3594-3611. The therapeutic effect may also be measured by improved motor skills, cognitive function, sensory perception, speech and/or a decrease in the propensity to seizure in the mammal undergoing treatment. Such functional/behavior tests used to assess sensorimotor and reflex function are described in, for example, Bederson et al. (1986) Stroke 17:472-476, DeRyck et al. (1992) Brain Res. 573:44-60, Markgraf et al. (1992) Brain Res. 575:238-246, Alexis et al. (1995) Stroke 26:2338-2346. Enhancement of neuronal survival may also be measured using the Scandinavian Stroke Scale (SSS) or the Barthel Index. Such assays can be readily used by one skilled in the art to determine the dosage range and/or appropriate regulatory agent of choice for the effective treatment of an ischemic event.

For purposes of regulating the development of a transplanted donor cell in a mammal, the therapeutically effective amount or dose of a regulatory agent is about 0.002 mg/kg to about 2.0 mg/kg of body weight or from about 0.03 mg/kg to about 0.6 mg/kg of body weight. Alternatively, the regulatory agent may be administered at 0.005, 0.007, 0.009, 0.01, 0.04, 0.06, 0.08, 0.1, 0.2, 0.4, 0.6, 0.8, 1.0, 1.2, 1.4, 1.6, 1.8, or 2.0 mg/kg of body weight. It is further recognized that a lower dose range of certain regulatory agents (i.e., ADNF) may be preferred. In these embodiments, the regulatory agent can be administered from about 0.1 ng/kg to about 20 ng/kg. Alternatively, the regulatory agent can be administered at 0.2, 0.4, 0.6, 0.8, 1, 2, 4, 8, 12, 15, 18, and 19 ng/kg of body weight.

These doses depend on factors including the efficiency with which the regulatory agent is transported from the nasal cavity or the tissue innervated by the trigeminal nerve to the CNS. A larger total dose can be delivered by multiple administrations of the agent.

Intermittent Dosing

In another embodiment of the invention, the pharmaceutical composition comprising the therapeutically effective dose of a regulatory agent is administered intermittently. By “intermittent administration” is intended administration of a therapeutically effective dose of a regulatory agent, followed by a time period of discontinuance, which is then followed by another administration of a therapeutically effective dose, and so forth. Administration of the therapeutically effective dose may be achieved in a continuous manner, as for example with a sustained-release formulation, or it may be achieved according to a desired daily dosage regimen, as for example with one, two, three, or more administrations per day. By “time period of discontinuance” is intended a discontinuing of the continuous sustained-released or daily administration of the regulatory agent. The time period of discontinuance may be longer or shorter than the period of continuous sustained-release or daily administration. During the time period of discontinuance, the regulatory agent level in the relevant tissue is substantially below the maximum level obtained during the treatment. The preferred length of the discontinuance period depends on the concentration of the effective dose and the form of regulatory agent used. The discontinuance period can be at least 2 days, preferably is at least 4 days, more preferably is at least 1 week and generally does not exceed a period of 4 weeks. When a sustained-release formulation is used, the discontinuance period must be extended to account for the greater residence time of regulatory agent at the site of injury. Alternatively, the frequency of administration of the effective dose of the sustained-release formulation can be decreased accordingly. An intermittent schedule of administration of regulatory agent can continue until the desired therapeutic effect, and ultimately treatment of the disease or disorder is achieved.

In yet another embodiment, intermittent administration of the therapeutically effective dose of regulatory agent is cyclic. By “cyclic” is intended intermittent administration accompanied by breaks in the administration, with cycles ranging from about 1 month to about 2, 3, 4, 5, or 6 months. For example, the administration schedule might be intermittent administration of the effective dose of regulatory agent, wherein a single short-term dose is given once per week for 4 weeks, followed by a break in intermittent administration for a period of 3 months, followed by intermittent administration by administration of a single short-term dose given once per week for 4 weeks, followed by a break in intermittent administration for a period of 3 months, and so forth. As another example, a single short-term dose may be given once per week for 2 weeks, followed by a break in intermittent administration for a period of 1 month, followed by a single short-term dose given once per week for 2 weeks, followed by a break in intermittent administration for a period of 1 month, and so forth. A cyclic intermittent schedule of administration of a regulatory agent to a subject may continue until the desired therapeutic effect, and ultimately treatment of the disorder or disease is achieved.

Pharmaceutical Composition

In addition to the therapeutically effective dose of a regulatory agent, the composition can include, for example, any pharmaceutically acceptable additive, carrier, and/or adjuvant that can promote the transfer of this agent within or through a tissue innervated by the trigeminal nerve or olfactory nerve or along or through a neural pathway. Alternatively, the composition can comprise a regulatory agent combined with substances that assist in transporting the regulatory agent to a transplanted donor cell. The composition can further comprise more than one regulatory agent so long as the therapeutic efficacy of the regulatory agent to augment the clinical outcome of the cell replacement strategy is not lessened.

By “pharmaceutically acceptable carrier” is intended a carrier that is conventionally used in the art to facilitate the storage, administration, and/or the biological activity of a regulatory agent. A carrier may also reduce any undesirable side effects of the regulatory agent. A suitable carrier should be stable, i.e., incapable of reacting with other ingredients in the formulation. It should not produce significant local or systemic adverse effect in recipients at the dosages and concentrations employed for treatment. Such carriers are generally known in the art.

Suitable carriers for this invention include those conventionally used for large stable macromolecules such as albumin, gelatin, collagen, polysaccharide, monosaccharides, polyvinylpyrrolidone, polylactic acid, polyglycolic acid, polymeric amino acids, fixed oils, ethyl oleate, liposomes, glucose, sucrose, lactose, mannose, dextrose, dextran, cellulose, mannitol, sorbitol, polyethylene glycol (PEG), and the like.

Water, saline, aqueous dextrose, and glycols are preferred liquid carriers, particularly (when isotonic) for solutions. The carrier can be selected from various oils, including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. The compositions can be subjected to conventional pharmaceutical expedients, such as sterilization, and can contain conventional pharmaceutical additives, such as preservatives, stabilizing agents, wetting, or emulsifying agents, salts for adjusting osmotic pressure, buffers, and the like.

Other acceptable components in the composition include, but are not limited to, isotonicity-modifying agents such as water, saline, and buffers including phosphate, citrate, succinate, acetic acid, and other organic acids or their salts. Typically, the pharmaceutically acceptable carrier also includes one or more stabilizers, reducing agents, anti-oxidants and/or anti-oxidant chelating agents. The use of buffers, stabilizers, reducing agents, anti-oxidants and chelating agents in the preparation of protein-based compositions, particularly pharmaceutical compositions, is well known in the art. See, Wang et al. (1980) J. Parent. Drug Assn. 34(6):452-462; Wang et al.(1988) J. Parent. Sci. Tech. 42:S4-S26 (Supplement); Lachman et al.(1968) Drug and Cosmetic Industry 102(1):36-38, 40, and 146-148; Akers (1988) J. Parent. Sci. Tech. 36(5):222-228; and Methods in Enzymology, Vol. XXV, ed. Colowick and Kaplan, “Reduction of Disulfide Bonds in Proteins with Dithiothreitol,” by Konigsberg, pp. 185-188.

Suitable buffers include acetate, adipate, benzoate, citrate, lactate, maleate, phosphate, tartarate, borate, tri(hydroxymethyl aminomethane), succinate, glycine, histidine, the salts of various amino acids, or the like, or combinations thereof. See Wang (1980) supra at page 455. Suitable salts and isotonicifiers include sodium chloride, dextrose, mannitol, sucrose, trehalose, or the like. Where the carrier is a liquid, it is preferred that the carrier is hypotonic or isotonic with oral, conjunctival, or dermal fluids and has a pH within the range of 4.5-8.5. Where the carrier is in powdered form, it is preferred that the carrier is also within an acceptable non-toxic pH range.

Suitable reducing agents, which maintain the reduction of reduced cysteines, include dithiothreitol (DTT also known as Cleland's reagent) or dithioerythritol at 0.01% to 0.1% wt/wt; acetylcysteine or cysteine at 0.1% to 0.5% (pH 2-3); and thioglycerol at 0.1% to 0.5% (pH 3.5 to 7.0) and glutathione. See Akers (1988) supra at pages 225-226. Suitable antioxidants include sodium bisulfite, sodium sulfite, sodium metabisulfite, sodium thiosulfate, sodium formaldehyde sulfoxylate, and ascorbic acid. See Akers (1988) supra at page 225. Suitable chelating agents, which chelate trace metals to prevent the trace metal catalyzed oxidation of reduced cysteines, include citrate, tartarate, ethylenediaminetetraacetic acid (EDTA) in its disodium, tetrasodium, and calcium disodium salts, and diethylenetriamine pentaacetic acid (DTPA). See, e.g., Wang (1980) supra at pages 457-458 and 460-461, and Akers (1988) supra at pages 224-227.

The composition can include one or more preservatives such as phenol, cresol, paraaminobenzoic acid, BDSA, sorbitrate, chlorhexidine, benzalkonium chloride, or the like. Suitable stabilizers include carbohydrates such as trehalose or glycerol. The composition can include a stabilizer such as one or more of microcrystalline cellulose, magnesium stearate, mannitol, or sucrose to stabilize, for example, the physical form of the composition; and one or more of glycine, arginine, hydrolyzed collagen, or protease inhibitors to stabilize, for example, the chemical structure of the composition. Suitable suspending agents include carboxymethyl cellulose, hydroxypropyl methylcellulose, hyaluronic acid, alginate, chondroitin sulfate, dextran, maltodextrin, dextran sulfate, or the like. The composition can include an emulsifier such as polysorbate 20, polysorbate 80, pluronic, triolein, soybean oil, lecithins, squalene and squalanes, sorbitan treioleate, or the like. The composition can include an antimicrobial such as phenylethyl alcohol, phenol, cresol, benzalkonium chloride, phenoxyethanol, chlorhexidine, thimerosol, or the like. Suitable thickeners include natural polysaccharides such as mannans, arabinans, alginate, hyaluronic acid, dextrose, or the like; and synthetic ones like the PEG hydrogels of low molecular weight; and aforementioned suspending agents.

The composition can include an adjuvant such as cetyl trimethyl ammonium bromide, BDSA, cholate, deoxycholate, polysorbate 20 and 80, fusidic acid, or the like. Suitable sugars include glycerol, threose, glucose, galactose, mannitol, and sorbitol.

Preferred compositions include one or more of a solubility enhancing additive, preferably a cyclodextrin; a hydrophilic additive, preferably a monosaccharide or oligosaccharide; an absorption promoting additive, preferably a cholate, a deoxycholate, a fusidic acid, or a chitosan; a cationic surfactant, preferably a cetyl trimethyl ammonium bromide; a viscosity enhancing additive, preferably to promote residence time of the composition at the site of administration, preferably a carboxymethyl cellulose, a maltodextrin, an alginic acid, a hyaluronic acid, or a chondroitin sulfate; or a sustained release matrix, preferably a polyanhydride, a polyorthoester, a hydrogel, a particulate slow release depo system, preferably a polylactide co-glycolides (PLG), a depo foam, a starch microsphere, or a cellulose derived buccal system; a lipid-based carrier, preferably an emulsion, a liposome, a niosome, or a micelle. The composition can include a bilayer destabilizing additive, preferably a phosphatidyl ethanolamine; a fusogenic additive, preferably a cholesterol hemisuccinate.

Other preferred compositions for sublingual administration including, for example, a bioadhesive to retain the regulatory agent sublingually; a spray, paint, or swab applied to the tongue; retaining a slow dissolving pill or lozenge under the tongue; or the like. Other preferred compositions for transdermal administration include a bioadhesive to retain the regulatory agent on or in the skin; a spray, paint, cosmetic, or swab applied to the skin; or the like.

These lists of carriers and additives are by no means complete, and a worker skilled in the art can choose excipients from the GRAS (generally regarded as safe) list of chemicals allowed in the pharmaceutical preparations and those that are currently allowed in topical and parenteral formulations.

For the purposes of this invention, the pharmaceutical composition comprising a regulatory agent can be formulated in a unit dosage and in a form such as a solution, suspension, or emulsion. The pharmaceutical composition to be administered to the tissue innervated by the trigeminal and/or olfactory neurons may be in the form of a powder, a granule, a solution, a cream, a spray (e.g., an aerosol), a gel, an ointment, an infusion, an injection, a drop, or a sustained-release composition, such as a polymer disk. Other forms of compositions for administration include a suspension of a particulate, such as an emulsion, a liposome, an insert that releases the regulatory agent slowly, and the like. The powder or granular forms of the pharmaceutical composition may be combined with a solution and with a diluting, dispersing, or surface active regulatory agent. Additional preferred compositions for administration include a bioadhesive to retain the regulatory agent or biologically active variant thereof at the site of administration; a spray, paint, or swab applied to the mucosa or epithelium; or the like. The composition can also be in the form of lyophilized powder, which can be converted into solution, suspension, or emulsion before administration. The pharmaceutical composition comprising at least one regulatory agent is preferably sterilized by membrane filtration and is stored in unit-dose or multi-dose containers such as sealed vials or ampoules.

The method for formulating a pharmaceutical composition is generally known in the art. A thorough discussion of formulation and selection of pharmaceutically acceptable carriers, stabilizers, and isomolytes can be found in Remington's Pharmaceutical Sciences (18^{th} ed.; Mack Publishing Company, Eaton, Pa., 1990), herein incorporated by reference.

The regulatory agent can also be formulated in a sustained-release form to prolong the presence of this pharmaceutically active component in the treated mammal, generally for longer than one day. Many methods of preparation of a sustained-release formulation are known in the art and are disclosed in Remington's Pharmaceutical Sciences (18^{th} ed.; Mack Publishing Company, Eaton, Pa., 1990), herein incorporated by reference.

Generally, a regulatory agent can be entrapped in semipermeable matrices of solid hydrophobic polymers. The matrices can be shaped into films or microcapsules. Examples of such matrices include, but are not limited to, polyesters, copolymers of L-glutamic acid and gamma ethyl-L-glutamate (Sidman et al. (1983) Biopolymers 22: 547-556), polylactides (U.S. Pat. No. 3,773,919 and EP 58,481), polylactate polyglycolate (PLGA) such as polylactide-co-glycolide (see, for example, U.S. Pat. Nos. 4,767,628 and 5,654,008), hydrogels (see, for example, Langer et al. (1981) J. Biomed. Mater. Res. 15:167-277; Langer (1982) Chem. Tech. 12:98-105), non-degradable ethylene-vinyl acetate (e.g., ethylene vinyl acetate disks and poly(ethylene-co-vinyl acetate)), degradable lactic acid-glycolic acid copolyers such as the Lupron Depot™, poly-D-(−)-3-hydroxybutyric acid (EP 133,988), hyaluronic acid gels (see, for example, U.S. Pat. No. 4,636,524), alginic acid suspensions, nanoparticles (see, for example, De et al. (2001) Artif. Cells Blood Substit. Immobil. Biotech. 29:31-46; Venugopalan et al. (2001) Pharmazie 56:217-219; and Zhang et al. (2001) Acc. Chem. Res. 34:249-256; all of which are herein incorporated by reference), and the like.

Suitable microcapsules can also include hydroxymethylcellulose or gelatin-microcapsules and polymethyl methacrylate microcapsules prepared by coacervation techniques or by interfacial polymerization. See International Publication No. WO 99/24061, entitled “Method for Producing Sustained-Release Formulations,” wherein proteins are encapsulated in PLGA microspheres, herein incorporated by reference. In addition, microemulsions or colloidal drug delivery systems such as liposomes and albumin microspheres, may also be used. See Remington's Pharmaceutical Sciences (18^{th} ed.; Mack Publishing Company Co., Eaton, Pa., 1990). Other sustained-release compositions employ a bioadhesive to retain the pharmacologically active agent at the site of administration. Other pharmaceutical compositions that may be useful in administering a regulatory agent of interest by the methods of the present invention include Captisol.

Among the optional substances that may be combined with regulatory agents in the pharmaceutical composition are. lipophilic substances that can enhance absorption of the regulatory agent through the mucosa or epithelium of the nasal cavity to damaged cells in the CNS. The regulatory agent may be mixed with a lipophilic adjuvant alone or in combination with a carrier, or may be combined with one or several types of micelle or liposome substances. Among the preferred lipophilic substances are cationic liposomes including one or more of phosphatidyl choline, lipofectin, DOTAP, or the like. These liposomes may include other lipophilic substances such as gangliosides and phosphatidylserine (PS). Also preferred are micellar additives such as GM-1 gangliosides and phosphatidylserine (PS), which may be combined with the regulatory agent either alone or in combination. GM-1 ganglioside can be included at 1-10 mole percent in any liposomal compositions or in higher amounts in micellar structures. Protein agents can be either encapsulated in particulate structures or incorporated as part of the hydrophobic portion of the structure depending on the hydrophobicity of the protein agent. One preferred liposomal formulation employs Depofoam. The neuroprotective agent can be encapsulated in multivesicular liposomes, as disclosed in the copending application entitled “High and Low Load Formulations of IGF-I in Multivesicular Liposomes,” International Publication No. WO 99/12522, herein incorporated by reference.

The pharmaceutical composition may additionally include a solubilizing compound to enhance stability of the regulatory agent or biologically active variant thereof. For IGF-I, a preferred solubilizing agent includes a guanidinium group that is capable of enhancing its solubility. Examples of such solubilizing compounds include the amino acid arginine, as well as amino acid analogs of arginine that retain the ability to enhance solubility of IGF-I or biologically active variant thereof at pH 5.5 or greater. Such analogs include, without limitation, dipeptides and tripeptides that contain arginine. By “enhancing the solubility” is intended increasing the amount of growth factor or biologically active variant thereof that can be dissolved in solution at pH 5.5 or greater in the presence of a guanidinium-containing compound compared to the amount of this protein that can be dissolved at pH 5.5 or greater in a solution with the same components but lacking the guanidinium-containing compound. The ability of a guanidinium-containing compound to enhance the solubility of the growth factor or biologically active variant thereof can be determined using methods well known in the art. In general, the concentration of the solubilizing compound present in the composition will be from about 10 mM to about 1 M, and, for example, in the case of the compound arginine, in a concentration range of about 20 mM to about 200 mM, as disclosed in the copending application entitled “Compositions Providing for Increased IGF-I Solubility,” International Publication No. WO 99/24063, herein incorporated by reference.

In one embodiment, the composition includes the combination of an effective amount of growth factor with poly(ethylene-co-vinyl acetate) to provide for controlled release of this growth factor.

A composition formulated for intranasal delivery may optionally comprise an odorant. An odorant agent is combined with the regulatory agent to provide an odorliferous sensation, and/or to encourage inhalation of the intranasal preparation to enhance delivery of a regulatory agent to the olfactory neuroepithelium. The odoriferous sensation provided by the odorant agent may be pleasant, obnoxious, or otherwise malodorous. The odorant receptor neurons are localized to the olfactory epithelium, which, in humans, occupies only a few square centimeters in the upper part of the nasal cavity. The cilia of the olfactory neuronal dendrites which contain the receptors are fairly long (about 30-200 um). A 10-30 um layer of mucus envelops the cilia that the odorant agent must penetrate to reach the receptors. See Snyder et al. (1988) J. Biol. Chem. 263: 13972-13974. Use of a lipophilic odorant agent having moderate to high affinity for odorant binding protein (OBP) is preferred. OBP has an affinity for small lipophilic molecules found in nasal secretions and may act as a carrier to enhance the transport of a lipophilic odorant substance and active regulatory agent to the olfactory receptor neurons. It is also preferred that an odorant agent is capable of associating with lipophilic additives such as liposomes and micelles within the preparation to further enhance delivery of the regulatory agent by means of OBP to the olfactory neuroepithelium. OBP may also bind directly to lipophilic agents to enhance transport of the regulatory agent to olfactory neural receptors.

Suitable odorants having a high affinity for OBP include terpanoids such as cetralva and citronellol, aldehydes such as amyl cinnamaldehyde and hexyl cinnamaldehyde, esters such as octyl isovalerate, jasmines such as C1S-jasmine and jasmal, and musk 89. Other suitable odorant agents include those which may be capable of stimulating odorant-sensitive enzymes such as adenylate cyslase and guanylate cyclase, or which may be capable of modifying ion channels within the olfactory system to enhance absorption of the regulatory agent.

The pharmaceutical composition having a unit dose of a regulatory agent can be, for example, in the form of solution, suspension, emulsion, or a sustained-release formulation. Preferably, the total volume of one dose of the pharmaceutical composition ranges from about 10 μl to about 0.2 ml, preferably from about 50 μl to about 200 μl. Alternatively, the total volume of one dose maybe about 10 μl, 50 μl, 100 μl, 150 μl, or 200 μl. It is apparent that the suitable volume can vary with factors such as the size of the nasal cavity to which the regulatory agent is administered and the solubility of the components in the composition.

It is recognized that the total amount of regulatory agent administered as a unit dose to a particular tissue will depend upon the type of pharmaceutical composition being administered, that is whether the composition is in the form of, for example, a solution, a suspension, an emulsion, or a sustained-release formulation. For example, where the pharmaceutical composition comprising a therapeutically effective amount of the regulatory agent is a sustained-release formulation, the regulatory agent is administered at a higher concentration. Needle-free subcutaneous administration to an extranasal tissue innervated by the trigeminal nerve may be accomplished by use of a device which employs a supersonic gas jet as a power source to accelerate an agent that is formulated as a powder or a microparticle into the skin. The characteristics of such a delivery method will be determined by the properties of the particle, the formulation of the agent and the gas dynamics of the delivery device. Similarly, the subcutaneous delivery of an aqueous composition can be accomplished in a needle-free manner by employing a gas-spring powered hand held device to produce a high force jet of fluid capable of penetrating the skin. Alternatively, a skin-patch formulated to mediate a sustained release of a composition can be employed for the transdermal delivery of a regulatory agent to a tissue innervated by the trigeminal nerve. Where the pharmaceutical composition comprises a therapeutically effective amount of an agent, or a combination of agents, in a sustained-release formulation, the agent(s) is/are administered at a higher concentration.

It should be apparent to a person skilled in the art that variations may be acceptable with respect to the therapeutically effective dose and frequency of the administration of the regulatory agent in this embodiment of the invention. The amount of the regulatory agent administered will be inversely correlated with the frequency of administration. Hence, an increase in the concentration of a regulatory agent in a single administered dose, or an increase in the mean residence time in the case of a sustained release form of the regulatory agent, generally will be coupled with a decrease in the frequency of administration.

It is recognized that a single dosage of the regulatory agent may be administered over the course of several minutes, hours, days, or weeks. A single dose of the regulatory agent may be sufficient. Alternatively, repeated doses may be given to a patient over the course of several hours, days or weeks. In addition, if desired, a combination of regulatory agents may be administered as noted elsewhere herein.

Further, the therapeutically effective amount or dose of a regulatory agent and the frequency of administration will depend on multiple factors including, for example, the neurologic disorder being treated, the severity of the neurologic disorder being treated, the size of the tissue encompassed by the donor cells, and the type of donor cell transplanted into the mammal and on the type of developmental regulation of the donor cell that is desired (i.e., potentiates the survival and/or proliferation of the transplanted donor cell; improves the capacity of the transplanted donor cell to establish synaptic connection with the host neurons and influences the developmental cues released by the transplanted donor cells).

Some minor degree of experimentation may be required to determine the most effective dose and frequency of dose administration, this being well within the capability of one skilled in the art once apprised of the present disclosure. The method of the present invention may be used with any mammal. Exemplary mammals include, but are not limited to rats, cats, dogs, horses, cows, sheep, pigs, and more preferably humans.

For purposes of regulating donor cell development and thereby reducing or preventing the clinical manifestation of the neurological disorder being treated, intranasal administration of one or more therapeutically effective doses of a regulatory agent may occur within minutes, hours, days, or even weeks of the initial transplantation of the donor host cell. For example, the initial therapeutic dose may be administered within about 2 to 4 hours, within about 2 to 6 hours, within about 8 hours, within about 10 hours, about 15 hours, about 24 hours, within about 36 hours, 48 hours, 72 hours, or about 96 hours following transplantation of the donor cell. One or more additional doses may be administered for hours, days, or weeks following the initial dose. Furthermore, the mammal undergoing a cell replacement regeneration therapy may be administered a regulatory agent within weeks, days, hours, or minutes prior to transplantation. Thus, for example, a mammal undergoing cell replacement therapy can be administered one or more therapeutically effective doses of regulatory agent thereof prior to, during, or following the surgical procedure.

Routes of Administration

The method of the invention administers the regulatory agent to tissue innervated by the trigeminal and olfactory nerves. Such nerve systems can provide a direct connection between the outside environment and the brain, thus providing advantageous delivery of a regulatory agent to the CNS, including brain, brain stem, and/or spinal cord. Regulatory agents are unable to cross or inefficiently cross the blood-brain barrier from the bloodstream into the brain. The methods of the present invention allow for the delivery of the regulatory agent by way of the olfactory and/or trigeminal nerve rather than through the circulatory system. This method of administration allows for the efficient delivery of a regulatory agent to the CNS, brain, or spinal cord. Furthermore, even for agents that do efficiently cross the blood brain barrier, administration via the trigeminal and olfactory nerves reduces systemic exposure and unwanted side effects.

The Olfactory Nerve

The method of the invention includes administration of a regulatory agent to tissue innervated by the olfactory nerve. The regulatory agent can be delivered to the olfactory area via delivery to the nasal cavity, preferably in the upper third of the nasal cavity or to the olfactory epithelium.

Fibers of the olfactory nerve are unmyelinated axons of olfactory receptor cells that are located in the superior one-third of the nasal mucosa. The olfactory receptor cells are bipolar neurons with swellings covered by hair-like cilia that project into the nasal cavity. At the other end, axons from these cells collect into aggregates and enter the cranial cavity at the roof of the nose. Surrounded by a thin tube of pia, the olfactory nerves cross the subarachnoid space containing CSF and enter the inferior aspects of the olfactory bulbs. Once the regulatory agent is dispensed into the nasal cavity, particularly to the upper third of nasal cavity, the regulatory agent can undergo transport through the nasal mucosa and into the olfactory bulb and other areas of the brain, such as the anterior olfactory nucleus, frontal cortex, hippocampal formation, amygdaloid nuclei, nucleus basalis of Meynert, hypothalamus, and the like.

The Trigeminal Nerve

The method of the invention administers the regulatory agent to tissue innervated by the trigeminal nerve. The trigeminal nerve innervates tissues of a mammal's (e.g., human) head including the tissues of the nasal cavity, skin of the face and scalp, oral tissues, and tissues of and surrounding the eye. The trigeminal nerve has three major branches, the ophthalmic nerve, the maxillary nerve, and the mandibular nerve. The method of the invention can administer the regulatory agent to tissue innervated by one or more of these branches.

The Ophthalmic Nerve and Its Branches

The method of the invention can administer the regulatory agent to tissue innervated by the ophthalmic nerve branch of the trigeminal nerve. The ophthalmic nerve innervates tissues including superficial and deep parts of the superior region of the face, such as the eye, the lacrimal gland, the conjunctiva, and skin of the scalp, forehead, upper eyelid, and nose.

The ophthalmic nerve has three branches known as the nasociliary nerve, the frontal nerve, and the lacrimal nerve. The method of the invention can administer the regulatory agent to tissue innervated by the one or more of the branches of the ophthalmic nerve. The frontal nerve and its branches innervate tissues including the upper eyelid, the scalp, particularly the front of the scalp, and the forehead, particularly the middle part of the forehead. The nasociliary nerve forms several branches including the long ciliary nerves, the ganglionic branches, the ethmoidal nerves, and the infratrochlear nerve. The long ciliary nerves innervate tissues including the eye. The posterior and anterior ethmoidal nerves innervate tissues including the ethmoidal sinus and the inferior two-thirds of the nasal cavity. The infratrochlear nerve innervates tissues including the upper eyelid and the lacrimal sack. The lacrimal nerve innervates tissues including the lacrimal gland, the conjunctiva, and the upper eyelid. Preferably, the present method administers the regulatory agent to the ethmoidal nerve.

The Maxillary Nerve and Its Branches

The method of the invention can administer the regulatory agent to tissue innervated by the maxillary nerve branch of the trigeminal nerve. The maxillary nerve innervates tissues including the roots of several teeth and facial skin, such as skin on the nose, the upper lip, the lower eyelid, over the cheekbone, over the temporal region. The maxillary nerve has branches including the infraorbital nerve, the zygomaticofacial nerve, the zygomaticotemporal nerve, the nasopalatine nerve, the greater palatine nerve, the posterior superior alveolar nerves, the middle superior alveolar nerve, and the interior superior alveolar nerve. The method of the invention can administer the regulatory agent to tissue innervated by the one or more of the branches of the maxillary nerve.

The infraorbital nerve innervates tissue including skin on the lateral aspect of the nose, upper lip, and lower eyelid. The zygomaticofacial nerve innervates tissues including skin of the face over the zygomatic bone (cheekbone). The zygomaticotemporal nerve innervates tissue including the skin over the temporal region. The posterior superior alveolar nerves innervate tissues including the maxillary sinus and the roots of the maxillary molar teeth. The middle superior alveolar nerve innervates tissues including the mucosa of the maxillary sinus, the roots of the maxillary premolar teeth, and the mesiobuccal root of the first molar tooth. The anterior superior alveolar nerve innervates tissues including the maxillary sinus, the nasal septum, and the roots of the maxillary central and lateral incisors and canine teeth. The nasopalantine nerve innervates tissues including the nasal septum. The greater palatine nerve innervates tissues including the lateral wall of the nasal cavity. Preferably, the present method administers the regulatory agent to the nasopalatine nerve and/or greater palatine nerve.

The Mandibular Nerve and Its Branches

The method of the invention can administer the regulatory agent to tissue innervated by the mandibular nerve branch of the trigeminal nerve. The mandibular nerve innervates tissues including the teeth, the gums, the floor of the oral cavity, the tongue, the cheek, the chin, the lower lip, tissues in and around the ear, the muscles of mastication, and skin including the temporal region, the lateral part of the scalp, and most of the lower part of the face.

The mandibular nerve has branches including the buccal nerve, the auriculotemporal nerve, the inferior alveolar nerve, and the lingual nerve. The method of the invention can administer the regulatory agent to one or more of the branches of the mandibular nerve. The buccal nerve innervates tissues including the cheek, particularly the skin of the cheek over the buccinator muscle and the mucous membrane lining the cheek, and the mandibular buccal gingiva (gum), in particular the posterior part of the buccal surface of the gingiva. The auriculotemporal nerve innervates tissues including the auricle, the external acoustic meatus, the tympanic membrane (eardrum), and skin in the temporal region, particularly the skin of the temple and the lateral part of the scalp. The inferior alveolar nerve innervates tissues including the mandibular teeth, in particular the incisor teeth, the gingiva adjacent the incisor teeth, the mucosa of the lower lip, the skin of the chin, the skin of the lower lip, and the labial mandibular gingivae. The lingual nerve innervates tissues including the tongue, particularly the anterior two-thirds of the tongue, the floor of the mouth, and the gingivae of the mandibular teeth. Preferably, the method of the invention administers the regulatory agent to one or more of the inferior alveolar nerve, the buccal nerve, and/or the lingual nerve.

Tissues Innervated by the Trigeminal Nerve

The method of the invention can administer the regulatory agent to any of a variety of tissues innervated by the trigeminal nerve. For example, the method can administer the regulatory agent to skin, epithelium, or mucosa of or around the face, the eye, the oral cavity, the nasal cavity, the sinus cavities, or the ear.

Preferably, the method of the invention administers the regulatory agent to skin innervated by the trigeminal nerve. For example, the present method can administer the regulatory agent to skin of the face, scalp, or temporal region. Suitable skin of the face includes skin of the chin; the upper lip, the lower lip; the forehead, particularly the middle part of the forehead; the nose, including the tip of the nose, the dorsum of the nose, and the lateral aspect of the nose; the cheek, particularly the skin of the cheek over the buccinator muscle or skin over the cheek bone; skin around the eye, particularly the upper eyelid and the lower eyelid; or a combination thereof. Suitable skin of the scalp includes the front of the scalp, scalp over the temporal region, the lateral part of the scalp, or a combination thereof. Suitable skin of the temporal region includes the temple and scalp over the temporal region.

Preferably, the method of the invention administers the regulatory agent to mucosa or epithelium innervated by the trigeminal nerve. For example, the present method can administer the regulatory agent to mucosa or epithelium of or surrounding the eye, such as mucosa or epithelium of the upper eyelid, the lower eyelid, the conjunctiva, the lacrimal system, or a combination thereof. The method of the invention can also administer the regulatory agent to mucosa or epithelium of the sinus cavities and/or nasal cavity, such as the inferior two-thirds of the nasal cavity and the nasal septum. The method of the invention can also administer the regulatory agent to mucosa or epithelium of the oral cavity, such as mucosa or epithelium of the tongue; particularly the anterior two-thirds of the tongue and under the tongue; the cheek; the lower lip; the upper lip; the floor of the oral cavity; the gingivae (gums), in particular the gingiva adjacent the incisor teeth, the labial mandibular gingivae, and the gingivae of the mandibular teeth; or a combination thereof. Preferably, the method of the invention administers the regulatory agent to mucosa or epithelium of the nasal cavity. Other preferred regions of mucosa or epithelium for administering the regulatory agent include the tongue, particularly sublingual mucosa or epithelium, the conjunctiva, the lacrimal system, particularly the palpebral portion of the lacrimal gland and the nasolacrimal ducts, the mucosa of the lower eyelid, the mucosa of the cheek, or a combination thereof.

Preferably, the method of the invention administers the regulatory agent to nasal tissues innervated by the trigeminal nerve. For example, the present method can administer the regulatory agent to nasal tissues including the sinuses, the inferior two-thirds of the nasal cavity and the nasal septum. Preferably, the nasal tissue for administering the regulatory agent includes the inferior two-thirds of the nasal cavity and the nasal septum.

Preferably, the method of the invention administers the regulatory agent to oral tissues innervated by the trigeminal nerve. For example, the present method can also administer the regulatory agent to oral tissues such as the teeth, the gums, the floor of the oral cavity, the cheeks, the lips, the tongue, particularly the anterior two-thirds of the tongue, or a combination thereof. Suitable teeth include mandibular teeth, such as the incisor teeth. Suitable portions of the teeth include the roots of several teeth, such as the roots of the maxillary molar teeth, the maxillary premolar teeth, the maxillary central and lateral incisors, the canine teeth, and the mesiobuccal root of the first molar tooth, or a combination thereof. Suitable portions of the lips include the skin and mucosa of the upper and lower lips. Suitable gums include the gingiva adjacent the incisor teeth and the gingivae of the mandibular teeth, such as the labial mandibular gingivae, or a combination thereof. Suitable portions of the cheek include the skin of the cheek over the buccinator muscle, the mucous membrane lining the cheek, and the mandibular buccal gingiva (gum), in particular the posterior part of the buccal surface of the gingiva, or a combination thereof. Preferred oral tissue for administering the regulatory agent includes the tongue, particularly sublingual mucosa or epithelium, the mucosa inside the lower lip, the mucosa of the cheek, or a combination thereof.

Preferably, the method of the invention administers the regulatory agent to a tissue of or around the eye that is innervated by the trigeminal nerve. For example, the present method can administer the regulatory agent to tissue including the eye, the conjunctiva, and the lacrimal gland including the lacrimal sack, the skin or mucosa of the upper or lower eyelid, or a combination thereof. Preferred tissue of or around the eye for administering the regulatory agent includes the conjunctiva, the lachrimal system, the skin or mucosa of the eyelid, or a combination thereof. Regulatory agent that is administered conjunctivally but not absorbed through the conjunctival mucosa can drain through nasolachrimal ducts into the nose, where it can be transported to the CNS, brain, and/or spinal cord as though it had been intranasally administered.

Preferably, the method of the invention administers the regulatory agent to a tissue of or around the ear that is innervated by the trigeminal nerve. For example, the present method can administer the regulatory agent to tissue including the auricle, the external acoustic meatus, the tympanic membrane (eardrum), and the skin in the temporal region, particularly the skin of the temple and the lateral part of the scalp, or a combination thereof. Preferred tissue of or around the ear for administering the regulatory agent includes the skin of the temple.

Neuronal Transport

One embodiment of the present method includes administration of the regulatory agent to the subject in a manner such that the regulatory agent is transported to the CNS, brain, and/or spinal cord along a neural pathway. A neural pathway includes transport within or along a neuron, through or by way of lymphatics running with a neuron, through or by way of a perivascular space of a blood vessel running with a neuron or neural pathway, through or by way of an adventitia of a blood vessel running with a neuron or neural pathway, or through an hemangiolymphatic system. The invention prefers transport of a regulatory agent by way of a neural pathway, rather than through the circulatory system, so that regulatory agents that are unable to, or only poorly, cross the blood-brain barrier from the bloodstream into the brain can be delivered to the lymphatic system, CNS, brain, and/or spinal cord. The regulatory agent, once past the blood-brain barrier and in the CNS, can then be delivered to various areas of the brain or spinal cord through lymphatic channels, through a perivascular space, or transport through or along neurons. In one embodiment, the regulatory agent preferably accumulates in areas having the greatest density of receptor or binding sites for that regulatory agent.

Use of a neural pathway to transport a regulatory agent to the brain, spinal cord, or other components of the central nervous system obviates the obstacle presented by the blood-brain barrier so that medications that cannot normally cross that barrier, can be delivered directly to the brain, cerebellum, brain stem, or spinal cord. Although the regulatory agent that is administered may be absorbed into the bloodstream as well as the neural pathway, the regulatory agent preferably provides minimal effects systemically. In addition, the invention can provide for delivery of a more concentrated level of the regulatory agent to neural cells since the regulatory agent does not become diluted in fluids present in the bloodstream. As such, the invention provides an improved method for delivering a regulatory agent to the CNS, brain, and/or spinal cord.

The Olfactory Neural Pathway

One embodiment of the present method includes delivery of the regulatory agent to the subject in a manner such that the regulatory agent is transported into the CNS, brain, and/or spinal cord along an olfactory neural pathway. Typically, such an embodiment includes administering the regulatory agent to tissue innervated by the olfactory nerve and inside the nasal cavity. The olfactory neural pathway innervates primarily the olfactory epithelium in the upper third of the nasal cavity, as described above. Application of the regulatory agent to a tissue innervated by the olfactory nerve can deliver the regulatory agent to damaged neurons or cells of the CNS, brain, and/or spinal cord. Olfactory neurons innervate this tissue and can provide a direct connection to the CNS, brain, and/or spinal cord due, it is believed, to their role in olfaction.

Delivery through the olfactory neural pathway can employ lymphatics that travel with the olfactory nerve to the various brain areas and from there into dural lymphatics associated with portions of the CNS, such as the spinal cord. Transport along the olfactory nerve can also deliver regulatory agents to an olfactory bulb. A perivascular pathway and/or a hemangiolymphatic pathway, such as lymphatic channels running within the adventitia of cerebral blood vessels, can provide an additional mechanism for transport of therapeutic regulatory agents to the brain and spinal cord from tissue innervated by the olfactory nerve.

A regulatory agent can be administered to the olfactory nerve, for example, through the olfactory epithelium. Such administration can employ extracellular or intracellular (e.g., transneuronal) anterograde and retrograde transport of the regulatory agent entering through the olfactory nerves to the brain and its meninges, to the brain stem, or to the spinal cord. Once the regulatory agent is dispensed into or onto tissue innervated by the olfactory nerve, the regulatory agent may transport through the tissue and travel along olfactory neurons into areas of the CNS including the brain stem, cerebellum, spinal cord, olfactory bulb, and cortical and subcortical structures.

Delivery through the olfactory neural pathway can employ movement of a regulatory agent into or across mucosa or epithelium into the olfactory nerve or into a lymphatic, a blood vessel perivascular space, a blood vessel adventitia, or a blood vessel lymphatic that travels with the olfactory nerve to the brain and from there into meningial lymphatics associated with portions of the CNS such as the spinal cord. Blood vessel lymphatics include lymphatic channels that are around the blood vessels on the outside of the blood vessels. This also is referred to as the hemangiolymphatic system. Introduction of a regulatory agent into the blood vessel lymphatics does not necessarily introduce the regulatory agent into the blood.

The Trigeminal Neural Pathway

One embodiment of the present method includes delivery of the regulatory agent to the subject in a manner such that the regulatory agent is transported into the CNS, brain, and/or spinal cord along a trigeminal neural pathway. Typically, such an embodiment includes administering the regulatory agent to tissue innervated by the trigeminal nerve including inside and outside the nasal cavity. The trigeminal neural pathway innervates various tissues of the head and face, as described above. In particular, the trigeminal nerve innervates the nasal, sinusoidal, oral and conjunctival mucosa or epithelium, and the skin of the face. Application of the regulatory agent to a tissue innervated by the trigeminal nerve can deliver the regulatory agent to damaged neurons or cells of the CNS, brain, and/or spinal cord to cells of the lymphatic system. Trigeminal neurons innervate these tissues and can provide a direct connection to the CNS, brain, and/or spinal cord due, it is believed, to their role in the common chemical sense including mechanical sensation, thermal sensation and nociception (for example detection of hot spices and of noxious chemicals).

Delivery through the trigeminal neural pathway can employ lymphatics that travel with the trigeminal nerve to the pons and other brain areas and from there into dural lymphatics associated with portions of the CNS, such as the spinal cord. Transport along the trigeminal nerve can also deliver regulatory agents to an olfactory bulb. A perivascular pathway and/or a hemangiolymphatic pathway, such as lymphatic channels running within the adventitia of cerebral blood vessels, can provide an additional mechanism for transport of therapeutic regulatory agents to the spinal cord from tissue innervated by the trigeminal nerve.

The trigeminal nerve includes large diameter axons, which mediate mechanical sensation, e.g., touch, and small diameter axons, which mediate pain and thermal sensation, both of whose cell bodies are located in the semilunar (or trigeminal) ganglion or the mesencephalic trigeminal nucleus in the midbrain. Certain portions of the trigeminal nerve extend into the nasal cavity, oral and conjunctival mucosa and/or epithelium. Other portions of the trigeminal nerve extend into the skin of the face, forehead, upper eyelid, lower eyelid, dorsum of the nose, side of the nose, upper lip, cheek, chin, scalp and teeth. Individual fibers of the trigeminal nerve collect into a large bundle, travel underneath the brain and enter the ventral aspect of the pons. A regulatory agent can be administered to the trigeminal nerve, for example, through the nasal cavity's, oral, lingual, and/or conjunctival mucosa and/or epithelium; or through the skin of the face, forehead, upper eyelid, lower eyelid, dorsum of the nose, side of the nose, upper lip, cheek, chin, scalp and teeth. Such administration can employ extracellular or intracellular (e.g., transneuronal) anterograde and retrograde transport of the regulatory agent entering through the trigeminal nerves to the brain and its meninges, to the brain stem, or to the spinal cord. Once the regulatory agent is dispensed into or onto tissue innervated by the trigeminal nerve, the regulatory agent may transport through the tissue and travel along trigeminal neurons into areas of the CNS including the brain stem, cerebellum, spinal cord, olfactory bulb, and cortical and subcortical structures.

Delivery through the trigeminal neural pathway can employ movement of a regulatory agent across skin, mucosa, or epithelium into the trigeminal nerve or into a lymphatic, a blood vessel perivascular space, a blood vessel adventitia, or a blood vessel lymphatic that travels with the trigeminal nerve to the pons and from there into meningial lymphatics associated with portions of the CNS such as the spinal cord. Blood vessel lymphatics include lymphatic channels that are around the blood vessels on the outside of the blood vessels. This also is referred to as the hemangiolymphatic system. Introduction of a regulatory agent into the blood vessel lymphatics does not necessarily introduce the regulatory agent into the blood.

Neural Pathways and Nasal Administration

In one embodiment, the method of the invention can employ delivery by a neural pathway, e.g., a trigeminal or olfactory neural pathway, after administration to the nasal cavity. Upon administration to the nasal cavity, delivery via the trigeminal neural pathway may employ movement of a regulatory agent through the nasal mucosa and/or epithelium to reach a trigeminal nerve or a perivascular and/or lymphatic channel that travels with the nerve. Upon administration to the nasal cavity, delivery via the olfactory neural pathway may employ movement of a regulatory agent through the nasal mucosa and/or epithelium to reach the olfactory nerve or a perivascular and/or lymphatic channel that travels with the nerve.

For example, the regulatory agent can be administered to the nasal cavity in a manner that employs extracellular or intracellular (e.g., transneuronal) anterograde and retrograde transport into and along the trigeminal and/or olfactory nerves to reach the brain, the brain stem, or the spinal cord. Once the regulatory agent is dispensed into or onto nasal mucosa and/or epithelium innervated by the trigeminal and/or olfactory nerve, the regulatory agent may transport through the nasal mucosa and/or epithelium and travel along trigeminal and/or olfactory neurons into areas of the CNS including the brain stem, cerebellum, spinal cord, olfactory bulb, and cortical and subcortical structures. Alternatively, administration to the nasal cavity can result in delivery of a regulatory agent into a blood vessel perivascular space or a lymphatic that travels with the trigeminal and/or olfactory nerve to the pons, olfactory bulb, and other brain areas, and from there into meningeal lymphatics associated with portions of the CNS such as the spinal cord. Transport along the trigeminal and/or olfactory nerve may also deliver regulatory agents administered to the nasal cavity to the olfactory bulb, midbrain, diencephalon, medulla, and cerebellum. A regulatory agent administered to the nasal cavity can enter the ventral dura of the brain and travel in lymphatic channels within the dura.

In addition, the method of the invention can be carried out in a way that employs a perivascular pathway and/or an hemangiolymphatic pathway, such as a lymphatic channel running within the adventitia of a cerebral blood vessel, to provide an additional mechanism for transport of regulatory agent to the spinal cord from the nasal mucosa and/or epithelium. A regulatory agent transported by the hemangiolymphatic pathway does not necessarily enter the circulation. Blood vessel lymphatics associated with the circle of Willis as well as blood vessels following the trigeminal and/or olfactory nerve can also be involved in the transport of the regulatory agent.

Administration to the nasal cavity employing a neural pathway can deliver a regulatory agent to the lymphatic system, brain stem, cerebellum, spinal cord, and cortical and subcortical structures. The regulatory agent alone may facilitate this movement into the CNS, brain, and/or spinal cord. Alternatively, the carrier or other transfer-promoting factors may assist in the transport of the regulatory agent into and along the trigeminal and/or olfactory neural pathway. Administration to the nasal cavity of a therapeutic regulatory agent can bypass the blood-brain barrier through a transport system from the nasal mucosa and/or epithelium to the brain and spinal cord.

Neural Pathways and Transdermal Administration

In one embodiment, the method of the invention can employ delivery by a neural pathway, e.g., a trigeminal neural pathway, after transdermal administration. Upon transdermal administration, delivery via the trigeminal neural pathway may employ movement of a regulatory agent through the skin to reach a trigeminal nerve or a perivascular and/or lymphatic channel that travels with the nerve.

For example, the regulatory agent can be administered transdermally in a manner that employs extracellular or intracellular (e.g., transneuronal) anterograde and retrograde transport into and along the trigeminal nerves to reach the brain, the brain stem, or the spinal cord. Once the regulatory agent is dispensed into or onto skin innervated by the trigeminal nerve, the regulatory agent may transport through the skin and travel along trigeminal neurons into areas of the CNS including the brain stem, cerebellum, spinal cord, olfactory bulb, and cortical and subcortical structures. Alternatively, transdermal administration can result in delivery of a regulatory agent into a blood vessel perivascular space or a lymphatic that travels with the trigeminal nerve to the pons, olfactory bulb, and other brain areas, and from there into meningeal lymphatics associated with portions of the CNS such as the spinal cord. Transport along the trigeminal nerve may also deliver transdermally administered regulatory agents to the olfactory bulb, midbrain, diencephalon, medulla and cerebellum. The ethmoidal branch of the trigeminal nerve enters the cribriform region. An transdermally administered regulatory agent can enter the ventral dura of the brain and travel in lymphatic channels within the dura.

In addition, the method of the invention can be carried out in a way that employs a perivascular pathway and/or an hemangiolymphatic pathway, such as a lymphatic channel running within the adventitia of a cerebral blood vessel, to provide an additional mechanism for transport of regulatory agent to the spinal cord from the skin. A regulatory agent transported by the hemangiolymphatic pathway does not necessarily enter the circulation. Blood vessel lymphatics associated with the circle of Willis as well as blood vessels following the trigeminal nerve can also be involved in the transport of the regulatory agent.

Transdermal administration employing a neural pathway can deliver a regulatory agent to the brain stem, cerebellum, spinal cord and cortical and subcortical structures. The regulatory agent alone may facilitate this movement into the CNS, brain, and/or spinal cord. Alternatively, the carrier or other transfer-promoting factors may assist in the transport of the regulatory agent into and along the trigeminal neural pathway. Transdermal administration of a therapeutic regulatory agent can bypass the blood-brain barrier through a transport system from the skin to the brain and spinal cord.

Neural Pathways and Sublingual Administration

In another embodiment, the method of the invention can employ delivery by a neural pathway, e.g., a trigeminal neural pathway, after sublingual administration. Upon sublingual administration, delivery via the trigeminal neural pathway may employ movement of a regulatory agent from under the tongue and across the lingual epithelium to reach a trigeminal nerve or a perivascular or lymphatic channel that travels with the nerve.

For example, the regulatory agent can be administered sublingually in a manner that employs extracellular or intracellular (e.g., transneuronal) anterograde and retrograde transport through the oral mucosa and then into and along the trigeminal nerves to reach the brain, the brain stem, or the spinal cord. Once the regulatory agent is administered sublingually, the regulatory agent may transport through the oral mucosa by means of the peripheral processes of trigeminal neurons into areas of the CNS including the brain stem, spinal cord and cortical and subcortical structures. Alternatively, sublingual administration can result in delivery of a regulatory agent into lymphatics that travel with the trigeminal nerve to the pons and other brain areas and from there into meningeal lymphatics associated with portions of the CNS such as the spinal cord. Transport along the trigeminal nerve may also deliver sublingually administered regulatory agents to the olfactory bulbs, midbrain, diencephalon, medulla and cerebellum. The ethmoidal branch of the trigeminal nerve enters the cribriform region. A sublingually administered regulatory agent can enter the ventral dura of the brain and travel in lymphatic channels within the dura.

In addition, the method of the invention can be carried out in a way that employs an hemangiolymphatic pathway, such as a lymphatic channel running within the adventitia of a cerebral blood vessel, to provide an additional mechanism for transport of a regulatory agent to the spinal cord from the oral submucosa. A regulatory agent transported by the hemangiolymphatic pathway does not necessarily enter the circulation. Blood vessel lymphatics associated with the circle of Willis as well as blood vessels following the trigeminal nerve can also be involved in the transport of the regulatory agent.

Sublingual administration employing a neural pathway can deliver a regulatory agent to the brain stem, cerebellum, spinal cord and cortical and subcortical structures. The regulatory agent alone may facilitate this movement into the CNS, brain, and/or spinal cord. Alternatively, the carrier or other transfer-promoting factors may assist in the transport of the regulatory agent into and along the trigeminal neural pathway. Sublingual administration of a therapeutic regulatory agent can bypass the blood-brain barrier through a transport system from the oral mucosa to the brain and spinal cord.

Neural Pathways and Conjunctival Administration

In another embodiment, the method of the invention can employ delivery by a neural pathway, e.g. a trigeminal neural pathway, after conjunctival administration. Upon conjunctival administration, delivery via the trigeminal neural pathway may employ movement of a regulatory agent from the conjunctiva through the conjunctival epithelium to reach the trigeminal nerves or lymphatic channels that travel with the nerve.

For example, the regulatory agent can be administered conjunctivally in a manner that employs extracellular or intracellular (e.g., transneuronal) anterograde and retrograde transport through the conjunctival mucosa and then into and along the trigeminal nerves to reach the brain, the brain stem, or the spinal cord. Once the regulatory agent is administered conjunctivally, the regulatory agent may transport through the conjunctival mucosa by means of the peripheral processes of trigeminal neurons into areas of the CNS including the brain stem, spinal cord and cortical and subcortical structures. Alternatively, conjunctival administration can result in delivery of a regulatory agent into lymphatics that travel with the trigeminal nerve to the pons and other brain areas and from there into meningeal lymphatics associated with portions of the CNS such as the spinal cord. Transport along the trigeminal nerve may also deliver conjunctivally administered regulatory agents to the olfactory bulbs, midbrain, diencephalon, medulla and cerebellum. The ethmoidal branch of the trigeminal nerve enters the cribriform region. An conjunctivally administered regulatory agent can enter the ventral dura of the brain and travel in lymphatic channels within the dura.

In addition, the method of the invention can be carried out in a way that employs an hemangiolymphatic pathway, such as a lymphatic channel running within the adventitia of cerebral blood vessel, to provide an additional mechanism for transport of a regulatory agent to the spinal cord from the conjunctival submucosa. A regulatory agent transported by the hemangiolymphatic pathway does not necessarily enter the circulation. Blood vessel lymphatics associated with the circle of Willis as well as blood vessels following the trigeminal nerve can also be involved in the transport of the regulatory agent.

Conjunctival administration employing a neural pathway can deliver a regulatory agent to the brain stem, cerebellum, spinal cord and cortical and subcortical structures. The regulatory agent alone may facilitate this movement into the CNS, brain, and/or spinal cord. Alternatively, the carrier or other transfer-promoting factors may assist in the transport of the regulatory agent into and along the trigeminal neural pathway. Conjunctival administration of a therapeutic regulatory agent can bypass the blood-brain barrier through a transport system from the conjunctival mucosa to the brain and spinal cord.

Articles and Methods of Manufacture

The present invention also includes an article of manufacture providing a regulatory agent for administration to the CNS, brain, and/or spinal cord. The article of manufacture can include a vial or other container that contains a composition suitable for the present method together with any carrier, either dried or in liquid form. The article of manufacture further includes instructions in the form of a label on the container and/or in the form of an insert included in a box in which the container is packaged, for the carrying out the method of the invention. The instructions can also be printed on the box in which the vial is packaged. The instructions contain information such as sufficient dosage and administration information so as to allow the subject or a worker in the field to administer the regulatory agent. It is anticipated that a worker in the field encompasses any doctor, nurse, technician, spouse, or other care-giver that might administer the regulatory agent. The regulatory agent can also be self-administered by the subject.

According to the invention, a regulatory agent can be used for manufacturing a regulatory agent composition or medicament suitable for intranasal, conjunctival, transdermal, and/or sublingual administration. For example, a liquid or solid composition can be manufactured in several ways, using conventional techniques. A liquid composition can be manufactured by dissolving a regulatory agent in a suitable solvent, such as water, at an appropriate pH, including buffers or other excipients, for example to form a solution described herein above.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended embodiments.

That which is claimed:

1. A method of regulating the development of a donor cell in the central nervous system of a mammal comprising
administering a composition comprising at least one regulatory agent to a tissue innervated by the trigeminal nerve, the olfactory nerve, or a combination thereof, wherein the regulatory agent is absorbed through the tissue and transported to the central nervous system of the mammal in an amount effective to regulate the development of the donor cell.

2. The method of claim 1, wherein the tissue comprises a nasal cavity tissue, a cojunctiva, an oral tissue, or a skin.

3. The method of claim 2, wherein administering the regulatory agent to the conjuctiva comprises administering the regulatory agent between a lower eyelid and an eye.

4. The method of claim 2, wherein administering the regulatory agent to the skin comprises administering the regulatory agent to a face, a forehead, an upper eyelid, a lower eyelid, a dorsum of the nose, a side of the nose, an upper lip, a cheek, a chin, a scalp, or a combination thereof.

5. The method of claim 2, wherein administering the regulatory agent to the oral tissue comprises sublingual administration.

6. The method of claim 1, wherein the regulatory agent is administered to the nasal cavity.

7. The method of claim 1, wherein the mammal has a central nervous system disorder.

8. The method of claim 7, wherein the central nervous system disorder is a neurodegenerative disorder.

9. The method of claim 8, wherein the neurodegenerative disorder is selected from the group consisting of epilepsy, Huntington disease, Parkinson's disease, ALS, and Alzheimer's disease.

10. The method of claim 8, wherein the neurodegenerative disorder is a demyelinating or dysmyelinating disorder.

11. The method of claim 10, wherein the demyelinating or dysmyelinating disorder is selected from the group consisting of Pelizaeus-Merbacher disease and multiple sclerosis.

12. The method of claim 7, wherein the central nervous system disorder is selected from the group consisting of injury, spinal cord injury, stroke, and ischemia.

13. The method of claim 1, wherein the regulatory agent comprises a growth factor or biologically active variant thereof.

14. The method of claim 13, wherein the growth factor is IGF-I, NGF, bFGF, or a biologically active variant thereof.

15. The method of claim 14, wherein the growth factor or biologically active variant thereof is from a human.

16. The method of claim 1, wherein the donor cell is a multipotent stem cell, a neural stem cell, or a neuronal progenitor cell.

17. The method of claim 1, wherein the donor cell is derived from a fetal tissue.

18. The method of claim 1, wherein the effective amount of the regulatory agent is from about 0.002 mg/kg to about 2.0 mg/kg of body weight.

19. The method of claim 1, wherein the effective amount of the regulatory agent is from about 0.03 mg/kg to about 0.6 mg/kg of body weight.

20. The method of claim 1, wherein the effective amount of the regulatory agent is from about 0.1 ng/kg to about 20 ng/kg of body weight.

21. The method of claim 1, wherein the regulatory agent modulates the immune response of the mammal.

22. The method of claim 21, wherein the regulatory agent is a cytokine or a biologically active variant thereof.

23. The method of claim 22, wherein the cytokine is selected from the group consisting of interferon-alpha (IFN-α), interferon-beta (IFN-β, interferon-gamma (IFN-γ), and a biologically active variant thereof.

24. A method of treating a CNS disorder in a mammal comprising
administering to said mammal a composition comprising a therapeutically effective amount of at least one regulatory agent, wherein said composition is administered to a tissue innervated by the trigeminal nerve, the olfactory nerve, or a combination thereof, wherein the regulatory agent is absorbed through the tissue and transported to the central nervous system of the mammal and wherein the central nervous system of said mammal comprises a transplanted donor cell.

25. The method of claim 24, wherein the tissue comprises a nasal cavity tissue, a conjunctiva, an oral tissue, or a skin.

26. The method of claim 25, wherein administering the regulatory agent to the conjunctiva comprises administering the regulatory agent between a lower eyelid and an eye.

27. The method of claim 25, wherein administering the regulatory agent to the skin comprises administering the regulatory agent to a face, a forehead, an upper eyelid, a lower eyelid, a dorsum of the nose, a side of the nose, an upper lip, a cheek, a chin, a scalp, or a combination thereof.

28. The method of claim 25, wherein administering the regulatory agent to the oral tissue comprises sublingual administration.

29. The method of claim 24, wherein the regulatory agent is administered to a nasal cavity.

30. The method of claim 24, wherein the CNS disorder is a neurodegenerative disorder.

31. The method of claim 30, wherein the neurodegenerative disorder is selected from the group consisting of epilepsy, Huntington disease, Parkinson's disease, ALS, and Alzheimer's disease.

32. The method of claim 30, wherein the neurodegenerative disorder is a demyelinating or dysmyelinating disorder.

33. The method of claim 32, wherein the demyelinating or dysmyelinating disorder is selected from the group consisting of Pelizaeus-Merzbacher disease and multiple sclerosis.

34. The method of claim 24, wherein the CNS disorder is selected from the group consisting of head injury, spinal cord injury, stroke, and ischemia.

35. The method of claim 24, wherein the regulatory agent is a growth factor or biologically active variant thereof.

36. The method of claim 35, wherein the growth factor is IGF-I, NGF, bFGF, or a biologically active variant thereof.

37. The method of claim 24, wherein the donor cell is a multipotent stem cell, a neural stem cell, or a neuronal progenitor cell.

38. The method of claim 24, wherein the donor cell is derived from a fetal tissue.

39. The method of claim 24, wherein the effective amount of the regulatory agent is from about 0.002 mg/kg body weight to about 2.0 mg/kg of body weight.

40. The method of claim 24, wherein the effective amount of the regulatory agent is from about 0.03 mg/kg to about 0.6 mg/kg of body weight.

41. The method of claim 24, wherein the effective amount of the regulatory agent is from about 0.1 ng/kg to about 20 ng/kg of body weight.

42. The method of claim 24, wherein the regulatory agent modulates the immune response of the mammal.

43. The method of claim 42, wherein the regulatory agent is a cytokine or a biologically active variant thereof.

44. The method of claim 43, wherein the cytokine selected from the group consists of IFN-α, IFN-β, IFN-γ, and a biologically active variant thereof.
